(19) United States
(12) Patent Application Publication
(10) Pub. No.: US 2024/0055122 A1
(43) Pub. Date: Feb. 15, 2024

(54) METHODS, SYSTEMS AND RELATED ASPECTS FOR REAL-TIME PREDICTION OF ADVERSE OUTCOMES USING MACHINE LEARNING AND HIGH-DIMENSIONAL CLINICAL DATA

(71) Applicant: THE JOHNS HOPKINS UNIVERSITY, Baltimore, MD (US)

(72) Inventors: Julie K. SHADE, Baltimore, MD (US); Ashish DOSHI, Baltimore, MD (US); Eric SUNG, Baltimore, MD (US); Allison HAYS, Baltimore, MD (US); Natalia A. TRAYANOVA, Baltimore, MD (US)

(73) Assignee: THE JOHNS HOPKINS UNIVERSITY, Baltimore, MD (US)

(21) Appl. No.: 18/257,925

(22) PCT Filed: Dec. 17, 2021
(86) PCT No.: PCT/US2021/064106

§ 371 Date: Jun. 16, 2023

Related U.S. Application Data
(60) Provisional application No. 63/127,867, filed on Dec. 18, 2020.

(51) Int. Cl. G16H 50/20; A61B 5/00; G16H 50/30; G16H 10/60

(52) U.S. Cl. CPC G16H 50/20; A61B 5/7275; A61B 5/7267; G16H 50/30; G16H 10/60

(57) ABSTRACT

Provided herein are methods of generating models for prognosing cardiovascular outcomes for monitored subjects infected with an etiologic agent (e.g., severe acute respiratory syndrome coronavirus-2 or another etiologic agent). Related methods, systems, and computer program products are also provided.

### CROSS-REFERENCE TO RELATED APPLICATIONS

This application claims priority to U.S. Provisional Patent Application Ser. No. 63/127,867, filed Dec. 18, 2020, the disclosure of which is incorporated herein by reference.

### STATEMENT OF GOVERNMENT SUPPORT

This invention was made using U.S. Government support under grant 2029603 awarded by the National Science Foundation. The U.S. Government has certain rights in this invention.

### BACKGROUND

Patients with COVID-19, the disease caused by the novel severe acute respiratory syndrome coronavirus 2 (SARS-CoV-2), often present with cardiovascular (CV) manifestations such as myocardial infarction, thromboembolism, and heart failure. Clinically overt cardiac injury or cardiomyopathy is reported in 8 to 33% of hospitalized patients and is associated with up to 50% mortality, but imaging studies suggest the true incidence of cardiac involvement in all persons infected with SARS-CoV-2 could be as high as 60%. Thromboembolic events are also frequently reported in severe COVID-19 and are associated with mortality; one study found that 70.1% of non-survivors and 0.6% of survivors met criteria for disseminated intravenous coagulation. Furthermore, thromboembolic complications are more pronounced in acute COVID-19 infection than in other viral illnesses, and include pulmonary embolus and ischemic stroke, which can be fatal and are a significant cause of morbidity even as the infection resolves. Despite the prevalence of thromboembolism and cardiac injury and their associations with poor outcomes, no approach currently exists to forecast adverse CV events in COVID-19 patients in real time.

Machine learning (ML) techniques are ideal for discovering patterns in high-dimensional biomedical data, especially when little is known about the underlying biophysical processes. ML is thus well-positioned for applications in COVID-19 and indeed has been employed in screening, contract tracing, drug development, and outbreak forecasting. ML approaches have been developed for prognostic assessment of hospitalized patients with COVID-19, including models which predict in-hospital mortality, progression to severe disease, and outcomes related to respiratory function. An ML model was also proposed for prediction of thromboembolic events but it required that all variables be present for all patients; did not provide dynamic risk updates, and was trained with data from only 76 patients. Thus far, prognostic ML models have relied on clinical data available at a single time-point, and have not accounted for the dynamic and difficult-to-predict course of the disease.

Accordingly, there is a need for additional methods, and related aspects, for prognosing cardiovascular outcomes for patients having etiologic agent (e.g., viral (e.g., COVID-19 and the like), bacterial, fungal, etc.) infections.

### SUMMARY

The present disclosure relates, in certain aspects, to methods, systems, and computer readable media of use in generating models for prognosing adverse outcomes (e.g., adverse cardiovascular (CV) outcomes, such as complications of severe acute respiratory syndrome coronavirus-2 (SARS-CoV-2) infections, etc.) for a monitored subject infected with an etiologic agent. These and other aspects will be apparent upon a complete review of the present disclosure, including the accompanying figures.

In one aspect, the present disclosure provides a method of generating a model for prognosing a cardiovascular (CV) outcome for a monitored subject infected with an etiologic agent at partially using a computer. The method includes generating, by the computer, a training database that comprises a first set of data values of a first plurality of dynamic and static clinical parameters associated with at least a first plurality of monitored reference subjects infected with the etiologic agent. The method also includes executing, by the computer, at least one variable selection algorithm to select at least a subset of the first plurality of dynamic and static clinical parameters to generate at least a first set of model parameters. In addition, the method also includes executing, by the computer, at least one classification algorithm to generate the model for prognosing the CV outcome using at least a subset of the first set of model parameters.

In another aspect, the present disclosure provides a method of generating a model for prognosing a cardiovascular (CV) outcome for a monitored subject infected with an etiologic agent at partially using a computer. The method includes generating, by the computer, a first set of data values of a first plurality of dynamic clinical parameters associated with at least a first plurality of monitored reference subjects infected with the etiologic agent, wherein at least a subset of the first set of data values comprises one or more time-series data values. The method also includes processing, by the computer, at least some of the first set of data values for at least some of the first plurality of monitored reference subjects infected with the etiologic agent using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features. The method also includes combining, by the computer, at least some of the first set of processed dynamic features with a second set of data values of a first plurality of static clinical parameters associated with at least some of the first plurality of monitored reference subjects infected with the etiologic agent for one or more of the time windows to produce at least a first set of combined features, In addition, the method also includes training, by the computer, at least one classifier using at least some of the first set of combined features, thereby generating the model for prognosing the CV outcome for the monitored subject infected with the etiologic agent.

In another aspect, the present disclosure provides a method of generating a model for prognosing a cardiovascular (CV) outcome for a monitored subject infected with severe acute respiratory syndrome coronavirus-2 (SARS-CoV-2) at partially using a computer. The method includes generating, by the computer, a training database that comprises a first set of data values of a first plurality of dynamic and static clinical parameters associated with at least a first plurality of monitored reference subjects infected with the SARS-CoV-2. The method also includes executing, by the computer, at least one variable selection algorithm to select at least a subset of the first plurality of dynamic and static clinical parameters to generate at least a first set of model parameters. In addition, the method also includes executing, by the computer, at least one classification algorithm to generate the model for prognosing the CV outcome using at least a subset of the first set of model parameters.

In another aspect, the present disclosure provides a method of generating a model for prognosing a cardiovascular (CV) outcome for a monitored subject infected with severe acute respiratory syndrome coronavirus-2 (SARS-CoV-2) at partially using a computer. The method includes generating, by the computer, a first set of data values of a first plurality of dynamic clinical parameters associated with at least a first plurality of monitored reference subjects infected with the SARS-CoV-2, wherein at least a subset of the first set of data values comprises one or more time-series data values. The method also includes processing, by the computer, at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features. The method also includes combining, by the computer, at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features. In addition, the method also includes training, by the computer, at least one classifier using at least some of the first set of combined features, thereby generating the model for prognosing the CV outcome for the monitored subject infected with the SARS-CoV-2.

In certain embodiments, the plurality of dynamic and static clinical parameters differs between at two of the reference subjects. In some embodiments, one or more of the data values in the first set of data values is absent for one or more of the plurality of reference subjects. In some embodiments, the methods include adding one or more additional values to the first set of data values and/or one or more additional dynamic and static clinical parameters to the training database and updating the model for prognosing the CV outcome. In some embodiments, the methods include adding a second set of data values of a second plurality of dynamic and static clinical parameters associated with at least a second plurality of reference subjects infected with the SARS-CoV-2 to the training database and updating the model for prognosing the CV outcome. In some embodiments, the methods include updating the model for prognosing the CV outcome in substantially real-time. In certain embodiments, the methods include training the model for prognosing the CV outcome using at least using a stochastic gradient descent method.

In some embodiments, the first plurality of dynamic and static clinical parameters comprises one or more time-series variables. In certain embodiments, the first plurality of dynamic and static clinical parameters comprises more than about 100 different parameters. In some embodiments, the dynamic clinical parameters comprise one or more variables selected from the group consisting of: a dynamic clinical parameter described herein or otherwise known to a person having ordinary skill in the art. In some embodiments, the static clinical parameters comprise one or more variables selected from the group consisting of: a static clinical parameter described herein or otherwise known to a person having ordinary skill in the art. In some embodiments, the dynamic clinical parameters comprise one or more time series features selected from the group consisting of: a short feature, a long feature, and an exponentially weighted decaying feature. In some of these embodiments, the short feature comprises a selected period of time prior to a given time point. In some of these embodiments, the long feature comprises an entire period to time during which a given reference subject is monitored, wherein corresponding data values are un-weighted. In some of these embodiments, the exponentially weighted decaying feature comprises an entire period to time during which a given reference subject is monitored, wherein corresponding data values are weighted.

In some embodiments, at least two values in the first set of data values are obtained at different time points from a given monitored reference subject. In some embodiments, the methods include pre-processing one or more of the first set of data values in one or more sliding time windows. In some embodiments, one or more of the first set of data values of the first plurality of dynamic and static clinical parameters associated with the first plurality of monitored reference subjects infected with the SARS-CoV-2 are obtained when a given reference subject is monitored as an in-patient reference subject. In some embodiments, one or more of the first set of data values of the first plurality of dynamic and static clinical parameters associated with the first plurality of monitored reference subjects infected with the SARS-CoV-2 are obtained when a given reference subject is monitored as an out-patient reference subject.

In certain embodiments, the method includes using the model for prognosing the CV outcome to prognose at least one CV outcome of a monitored test subject infected with the SARS-CoV-2 at one or more time points to produce at least one prognosed test subject CV outcome. In certain embodiments, the method includes determining at least one test risk score for the test subject at the one or more time points, wherein a given test risk score that exceeds a predetermined threshold risk score indicates a probability of the test subject experiencing the CV outcome in a given time window beyond the one or more time points. In certain embodiments, the method includes determining the test risk score for the test subject in substantially real time. In certain embodiments, the method includes repeatedly updating the test risk score for the test subject during at least one selected period of time. In certain embodiments, the method includes integrating the test risk score into an electronic health record (EHR) for the test subject. In certain embodiments, the method includes administering one or more therapies to the monitored test subject in view of the prognosed test subject CV outcome.

In some embodiments, the CV outcome comprises one or more outcomes selected from the group consisting of: a CV outcome described herein or otherwise known to a person having ordinary skill in the art. In some embodiments, the variable selection algorithm is selected from the group consisting of: a supervised machine learning algorithm, an unsupervised machine learning algorithm, Incremental Association Markov Blanket algorithm, a Grow-Shrink algorithm, and a Semi-Interleaved Hiton-PC algorithm. In some embodiments, the classification algorithm is selected from the group consisting of: a random forest model, a classification and regression tree model, a linear discriminant analysis model, a decision tree learning model, a support vector machine, a nearest neighbor model, a logistic regression algorithm, an artificial neural network, a generated linear model, and a Bayesian model.

In another aspect, the present disclosure provides a system, comprising at least one controller that comprises, or is capable of accessing, computer readable media comprising non-transitory computer executable instructions which, when executed by at least one electronic processor, perform at least: generating a training database that comprises a first set of data values of a first plurality of dynamic and static clinical parameters associated with at least a first plurality of monitored reference subjects infected with an etiologic agent; executing at least one variable selection algorithm to select at least a subset of the first plurality of dynamic and static clinical parameters to generate at least a first set of model parameters; and executing at least one classification algorithm to generate the model for prognosing a cardiovascular (CV) outcome using at least a subset of the first set of model parameters.

In another aspect, the present disclosure provides a system, comprising at least one controller that comprises, or is capable of accessing, computer readable media comprising non-transitory computer executable instructions which, when executed by at least one electronic processor, perform at least: generating a first set of data values of a first plurality of dynamic clinical parameters associated with at least a first plurality of monitored reference subjects infected with an etiologic agent, wherein at least a subset of the first set of data values comprises one or more time-series data values; processing at least some of the first set of data values for at least some of the first plurality of monitored reference subjects infected with the etiologic agent using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features; combining at least some of the first set of processed dynamic features with a second set of data values of a first plurality of static clinical parameters associated with at least some of the first plurality of monitored reference subjects infected with the etiologic agent for one or more of the time windows to produce at least a first set of combined features; and training, by the computer, at least one classifier using at least some of the first set of combined features, thereby generating the model for prognosing a cardiovascular (CV) outcome for the monitored subject infected with the etiologic agent.

In another aspect, the present disclosure provides a system, comprising at least one controller that comprises, or is capable of accessing, computer readable media comprising non-transitory computer executable instructions which, when executed by at least one electronic processor, perform at least: generating a training database that comprises a first set of data values of a first plurality of dynamic and static clinical parameters associated with at least a first plurality of monitored reference subjects infected with severe acute respiratory syndrome coronavirus-2 (SARS-CoV-2); executing at least one variable selection algorithm to select at least a subset of the first plurality of dynamic and static clinical parameters to generate at least a first set of model parameters; and executing at least one classification algorithm to generate the model for prognosing a cardiovascular (CV) outcome using at least a subset of the first set of model parameters.

In another aspect, the present disclosure provides a system, comprising at least one controller that comprises, or is capable of accessing, computer readable media comprising non-transitory computer executable instructions which, when executed by at least one electronic processor, perform at least: generating a first set of data values of a first plurality of dynamic clinical parameters associated with at least a first plurality of monitored reference subjects infected with severe acute respiratory syndrome coronavirus-2 (SARS-CoV-2), wherein at least a subset of the first set of data values comprises one or more time-series data values; processing at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features; combining at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features; and training, by the computer, at least one classifier using at least some of the first set of combined features, thereby generating the model for prognosing a cardiovascular (CV) outcome for the monitored subject infected with the SARS-CoV-2.

In another aspect, the present disclosure provides a computer readable media comprising non-transitory computer executable instruction which, when executed by at least one electronic processor perform at least: generating a training database that comprises a first set of data values of a first plurality of dynamic and static clinical parameters associated with at least a first plurality of monitored reference subjects infected with an etiologic agent; executing at least one variable selection algorithm to select at least a subset of the first plurality of dynamic and static clinical parameters to generate at least a first set of model parameters; and executing at least one classification algorithm to generate the model for prognosing a cardiovascular (CV) outcome using at least a subset of the first set of model parameters.

In another aspect, the present disclosure provides a computer readable media comprising non-transitory computer executable instruction which, when executed by at least one electronic processor perform at least: generating a first set of data values of a first plurality of dynamic clinical parameters associated with at least a first plurality of monitored reference subjects infected with an etiologic agent, wherein at least a subset of the first set of data values comprises one or more time-series data values; processing at least some of the first set of data values for at least some of the first plurality of monitored reference subjects infected with the etiologic agent using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features; combining at least some of the first set of processed dynamic features with a second set of data values of a first plurality of static clinical parameters associated with at least some of the first plurality of monitored reference subjects infected with the etiologic agent for one or more of the time windows to produce at least a first set of combined features; and training, by the computer, at least one classifier using at least some of the first set of combined features, thereby generating the model for prognosing a cardiovascular (CV) outcome for the monitored subject infected with the etiologic agent.

In another aspect, the present disclosure provides a computer readable media comprising non-transitory computer executable instruction which, when executed by at least one electronic processor perform at least: generating a training database that comprises a first set of data values of a first plurality of dynamic and static clinical parameters associated with at least a first plurality of monitored reference subjects infected with severe acute respiratory syndrome coronavirus-2 (SARS-CoV-2); executing at least one variable selection algorithm to select at least a subset of the first plurality of dynamic and static clinical parameters to generate at least a first set of model parameters; and executing at least one classification algorithm to generate the model for prognosing a cardiovascular (CV) outcome using at least a subset of the first set of model parameters.

In another aspect, the present disclosure provides a computer readable media comprising non-transitory computer executable instruction which, when executed by at least one electronic processor perform at least: generating a first set of data values of a first plurality of dynamic clinical parameters associated with at least a first plurality of monitored reference subjects infected with severe acute respiratory syndrome coronavirus-2 (SARS-CoV-2), wherein at least a subset of the first set of data values comprises one or more time-series data values; processing at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features; combining at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features; and training, by the computer, at least one classifier using at least some of the first set of combined features, thereby generating the model for prognosing a cardiovascular (CV) outcome for the monitored subject infected with the SARS-CoV-2.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps according to some aspects disclosed herein.

FIG. 2 is a flow chart that schematically depicts exemplary method steps according to some aspects disclosed herein.

FIG. 3 is a schematic diagram of an exemplary system suitable for use with certain aspects disclosed herein.

FIG. 4: Schematic Overview of COVID-HEART Study. (A) Time-series clinical data used as input. Data shown here are representative and do not correspond with the risk score shown in (D). (B) Dynamic features pre-processing with sliding time windows. Relative intensity levels within the three feature windows represent the weighting of values at each time; darker colors indicate higher weight. (C) Combined features. For each time window, the processed dynamic features are combined with static features including demographics and comorbidities. Outcome labels are assigned per-window. (D) Continuously-updating risk score. The COVID-heart predictor provides a risk score (probability) for a given cardiovascular outcome in the K hours following a given time point. Shown is a sample risk score for a patient that experienced an event: green color indicates low risk score; yellow indicates a risk score within a pre-determined range of a threshold value, and the red indicates that the patient is at high risk for an event in the following K hours.

FIG. 5: Participant flow diagram for retrospective study of COVID-HEART. Inclusion and exclusion criteria were applied separately for prediction of each outcome. The data were then temporally divided into development and test sets as shown.

FIG. 6: The COVID-HEART predictor can accurately predict the risk of cardiac arrest and thromboembolic events in real time. (A) COVID-HEART 5-fold cross-validation performance metrics for the two CV outcomes: cardiac arrest and thromboembolic events. Values shown are the mean [95% confidence interval] for each metric over 20 full iterations of cross-validation. Cardiac arrest predictions presented here are for an outcome window of 2 hours, short-time feature window of 2 hours, and time-step of 1 hour. Thromboembolic event predictions shown here are for an outcome window of 24 hours, short-time feature window of 24 hours, and time-step of 24 hours. The best-performing classifier for prediction of each CV outcome is bolded. These were selected based on the area under the receiver operating characteristic curve (AUROC). (B) COVID-HEART test performance metrics for temporally divided test set. Characteristics of this set are provided in Supplementary Table 4. (C) COVID-HEART test performance metrics over 20 iterations of repeated temporally divided testing. (D) Risk of cardiac arrest prediction. Cross-validation (purple) and testing (orange) receiver operating characteristic (ROC) curves for prediction of cardiac arrest using the optimal classifier configuration: a linear classifier with all feature types. To generate the ROC curves, 20 iterations of 5-fold temporal patient-based cross validation were run resulting in a total of 20 test sets and 100 internal loops of cross-validation. Shaded regions represent the 95% confidence interval of each ROC curve. (E) Risk of thromboembolic event prediction.

FIG. 7: Examples of “true positive” predictions for two different patients, one from the cardiac arrest test set and one from the thromboembolic event test set. (A) Clinical time-series inputs (top 7 rows) from which the features with the largest coefficients were derived for prediction of cardiac arrest, and time-series risk score (bottom row) for a patient who experienced cardiac arrest during their hospitalization, and for whom the classifier's prediction was correct prior to the cardiac arrest. The most important features derived from these inputs are listed in Table 2. A new prediction is generated every hour. The x-axis refers to the days of admission relative to midnight on the first full day of admission. The binary risk threshold is 0.0008; the red bar indicates the hour during which the patient experienced cardiac arrest. Units for each predictor are as follows: WBC (cells/mm³), Pulse O₂ saturation (%), Pulse (beats/minutes), Chloride (mEq/L), CRP (mg/L), DBP (mmHg), SBP (mmHg). (B) Clinical time-series inputs (top 4 rows) from which the selected features were derived for prediction of thromboembolic events, and time-series risk score (bottom row) for a patient who experienced a thromboembolic event during their hospitalization. The most important features derived from these inputs are listed in Table 2. A new prediction is generated every 24 hours. The x-axis refers to the days of admission relative to midnight on the first full day of admission. Note that for all dynamic clinical data, values are assumed constant until a new measurement is made. Dashed line (bottom row) indicates binary risk threshold, determined by the development data; red bar indicates the day on which the patient experienced an imaging-confirmed thromboembolic event. Units for each predictor are as follows: magnesium (mEq/L), D-dimer (nmol/L), WBC (cells/mm³), IG Count (%). Abbreviations: white blood cell count (WBC), c-reactive protein (CRP), diastolic blood pressure (DBP), systolic blood pressure (SBP), immature granulocyte (IG).

FIG. 8: COVID-HEART cross-validation and testing results for outcome windows of different duration in predicting each CV outcome using the optimal classifier. Results for 5-fold stratified patient-based cross-validation and temporally separate test set for prediction of cardiac arrest (top) and thromboembolic events (bottom) within a given outcome window using the optimal classifier configuration from FIG. 6. Short feature window is 2 hours for prediction of cardiac arrest and 24 hours for prediction of thromboembolic events. Note comparable validation and test results, which indicates strong generalizability. Results shown are for the full temporally-divided development and validation sets (Supplementary Table 4).

FIG. 9: Two examples of “true negative” predictions for two patients, one from the cardiac arrest test set and one from the thromboembolic event test set, using the COVID-HEART predictor. (A) Clinical time-series inputs (top 7 rows) from which the features with the largest coefficients were derived for prediction of cardiac arrest, and time-series risk score (bottom row) for a patient who experienced cardiac arrest during their hospitalization, and for whom the classifier's prediction was correct prior to the cardiac arrest. The most important features derived from these inputs are listed in Table 2. A new prediction is generated every hour. The risk score is below 0.08% for the entire duration of the patient's admission. The date refers to the days of admission relative to midnight on the first full day of admission. Dashed line (bottom row) indicates binary risk threshold, determined by the development data. Units for each predictor are as follows: WBC (cells/mm³), Pulse O₂ saturation (%), Pulse (beats/minutes), Chloride (mEq/L), CRP (mg/L), DBP (mmHg), SBP (mmHg). (B) Clinical time-series inputs (top 4 rows) from which the selected features were derived for prediction of thromboembolic events, and time-series risk score (bottom row) for a patient who experienced a thromboembolic event during their hospitalization. The most important features derived from these inputs are listed in Table 2. A new prediction is generated every 24 hours. The risk score is low for the entire duration of the patient's admission. The x-axis refers to the days of admission relative to midnight on the first full day of admission. Note that for all dynamic clinical data, values are assumed constant until a new measurement is made. The binary risk threshold is 0.0024 and is not visible due to y-axis limits. Units for each predictor are as follows: magnesium (mEq/L), D-dimer (nmol/L), WBC (cells/mm³), IG Count (%). Abbreviations: white blood cell count (WBC), c-reactive protein (CRP), diastolic blood pressure (DBP), systolic blood pressure (SBP), immature granulocyte (IG).

FIG. 10: Investigation of incorrect predictions by the COVID-HEART predictor for two patients, one from the cardiac arrest test set and one from the thromboembolic event test set. (A) Clinical time-series inputs (top 7 rows) from which the features with the largest coefficients were derived for prediction of cardiac arrest, and time-series risk score (bottom row) for a patient who experienced cardiac arrest during their hospitalization, and for whom the classifier's prediction was correct prior to the cardiac arrest. The most important features derived from these inputs are listed in Table 2. A new prediction is generated every hour. The risk score fluctuates throughout the patient's hospitalization, crossing above the binary risk threshold several times. Dashed line (bottom row) indicates binary risk threshold, determined by the development data; red bar indicates the hour during which the patient experienced cardiac arrest. Units for each predictor are as follows: WBC (cells/mm³), Pulse O₂ saturation (%), Pulse (beats/minutes), Chloride (mEq/L), CRP (mg/L), DBP (mmHg), SBP (mmHg). (B) Clinical time-series inputs (top 4 rows) from which the selected features were derived for prediction of thromboembolic events, and time-series risk score (bottom row) for a patient who experienced a thromboembolic event during their hospitalization. The most important features derived from these inputs are listed in Table 2. A new prediction is generated every 24 hours. The risk score peaks midway through patient's hospitalization, then hovers around the binary risk threshold until the event. The x-axis refers to the days of admission relative to midnight on the first full day of admission. Note that for all dynamic clinical data, values are assumed constant until a new measurement is made. Dashed line (bottom row) indicates binary risk threshold, determined by the development data; red bar indicates the day on which the patient experienced an imaging-confirmed thromboembolic event. Units for each predictor are as follows: magnesium (mEq/L), D-dimer (nmol/L), WBC (cells/mm³), IG Count (%). Abbreviations: white blood cell count (WBC), c-reactive protein (CRP), diastolic blood pressure (DBP), systolic blood pressure (SBP), immature granulocyte (IG).

FIG. 11: More time windows are predicted positive for patients that eventually experience each outcome than patients who do not. Proportion of time windows predicted positive (risk probability greater than the binary risk threshold determined by the development data) for patients that do (solid line) and do not (dashed line) experience cardiac arrest (top) and thromboembolic events (bottom) in 5-fold patient-based cross-validation and in the separate test set. Results shown are for the full development and validation sets (Supplementary Table 4).

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, computer readable media, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., known ocular or other pathology and/or the like).

### Detailed Description

Cardiovascular (CV) manifestations of COVID-19 are of significant clinical concern. Current risk prediction for CV complications in COVID-19 is limited and existing approaches fail to account for the dynamic course of the disease. Here, we develop and validate the COVID-HEART predictor, a novel continuously-updating risk prediction technology to forecast CV complications in hospitalized patients with COVID-19. In some embodiments, the risk predictor is trained and tested with retrospective registry data from 2178 patients to predict two outcomes: cardiac arrest and imaging-confirmed thromboembolic events. In validating the model in these embodiments, we show that it can predict cardiac arrest with a median early warning time of 24 hours and an AUROC of 0.93, and thromboembolic events with a median early warning time of 72 hours and an AUROC of 0.71. The COVID-HEART predictor provides tangible clinical decision support in triaging patients and optimizing resource utilization, with its clinical utility extending well beyond COVID-19.

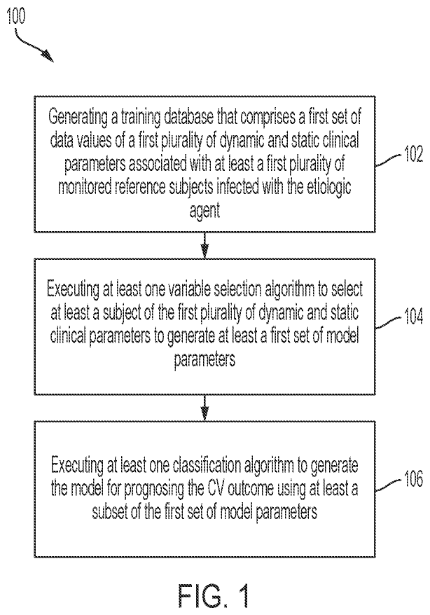

To illustrate, FIG. 1 is a flow chart that schematically depicts exemplary method steps of generating a model for prognosing a cardiovascular (CV) outcome for a monitored subject infected with an etiologic agent (e.g., a virus (e.g., SARS-CoV-2), a bacteria, a fungus, or the like). As shown, method 100 includes generating a training database that comprises a first set of data values of a first plurality of dynamic and static clinical parameters associated with at least a first plurality of monitored reference subjects infected with the etiologic agent (step 102). Method 100 also includes executing at least one variable selection algorithm to select at least a subset of the first plurality of dynamic and static clinical parameters to generate at least a first set of model parameters (step 104). In addition, method 100 also includes executing at least one classification algorithm to generate the model for prognosing the CV outcome using at least a subset of the first set of model parameters (step 106).

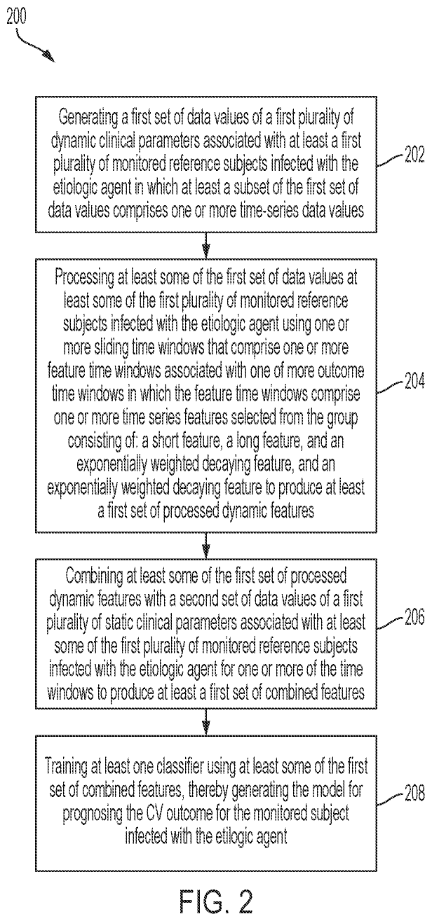

To further illustrate, FIG. 2 is a flow chart that schematically depicts some exemplary method steps of generating a model for prognosing a cardiovascular (CV) outcome for a monitored subject infected with an etiologic agent (e.g., a virus (e.g., SARS-CoV-2), a bacteria, a fungus, or the like). As shown, method 200 includes generating a first set of data values of a first plurality of dynamic clinical parameters associated with at least a first plurality of monitored reference subjects infected with the etiologic agent in which at least a subset of the first set of data values comprises one or more time-series data values (step 202). Method 200 also includes processing at least some of the first set of data values for at least some of the first plurality of monitored reference subjects infected with the etiologic agent using one or more sliding time windows that comprise one or more feature time windows associated with one or more outcome time windows in which the feature time windows comprise one or more time series features selected from the group consisting of: a short feature, a long feature, and an exponentially weighted decaying feature to produce at least a first set of processed dynamic features (step 204). Method 200 also includes combining at least some of the first set of processed dynamic features with a second set of data values of a first plurality of static clinical parameters associated with at least some of the first plurality of monitored reference subjects infected with the etiologic agent for one or more of the time windows to produce at least a first set of combined features (step 206). In addition, method 200 also includes training at least one classifier using at least some of the first set of combined features, thereby generating the model for prognosing the CV outcome for the monitored subject infected with the etiologic agent (step 208).

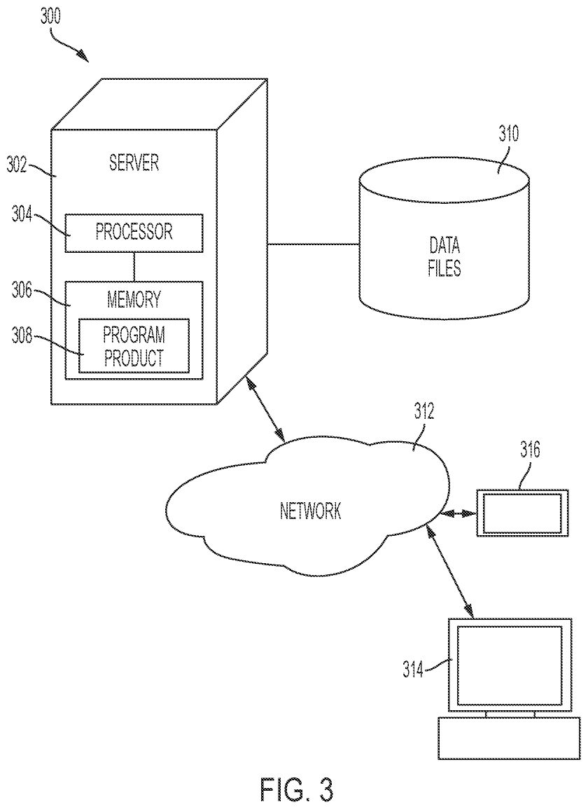

The present disclosure also provides various deep learning systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314, 316, (e.g., client-side computer terminals, telephones, tablets, laptops, other mobile devices, etc. (e.g., for receiving captured images and/or videos for further analysis, etc.)) positioned remote from camera device 318, and in communication with the remote server 302, through electronic communication network 312, such as the Internet or other internetwork. Communication devices 314, 316 typically include an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 (e.g., related to an ocular pathology model) stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon (e.g., entries corresponding to more reference images and/or videos, indexed therapies, etc.) searchable either directly or through search engine server 302. System 300 optionally also includes one or more other servers positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314, 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 508 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one captured image, EMR, and/or the like to be displayed (e.g., via communication devices 314, 316 or the like) and/or receive information from other system components and/or from a system user (e.g., via communication devices 314, 316, or the like).

In some aspects, program product 308 includes non-transitory computer-executable instructions which, when executed by electronic processor 304 perform at least: generating a training database that comprises a first set of data values of a first plurality of dynamic and static clinical parameters associated with at least a first plurality of monitored reference subjects infected with the etiologic agent, executing at least one variable selection algorithm to select at least a subset of the first plurality of dynamic and static clinical parameters to generate at least a first set of model parameters, and executing at least one classification algorithm to generate the model for prognosing the CV outcome using at least a subset of the first set of model parameters. Other exemplary executable instructions that are optionally performed are described further herein.

### Example: Real-Time Prediction of Cardiovascular Complications in Hospitalized Patients with Covid-19

Introduction

In this study, we develop and validate the first prognostic ML model to forecast the real-time risk of CV complications in hospitalized patients with COVID-19. We term the model the COVID-HEART predictor. We focus on predicting two clinically important CV outcomes in COVID-19: in-hospital cardiac arrest and thromboembolic events. In-hospital cardiac arrest is a clearly identifiable outcome and is often CV-related, thus it was selected to demonstrate the potential utility of COVID-HEART. Thromboembolic events are more difficult to identify and require imaging confirmation, thus, this outcome was selected to demonstrate the versatility of COVID-HEART in analyzing real-world clinical data and handling CV-specific outcomes. Finally, the predictor is tested in two different ways. First, it is tested with data from patients hospitalized after the end of data collection for patients in the development set, to ascertain that COVID-HEART can accurately predict risk in real time for new patients in the face of rapidly changing clinical treatment guidelines. The predictor is next tested with leave-hospital-out nested cross-validation to assess its performance when training and testing is done with data from different populations.

Materials and Methods

Patient Population

The COVID-HEART predictor was developed and validated in a retrospective cohort study approved by the Johns Hopkins University Institutional Review Board on May 21, 2020 under protocol number IRB00249548: Prediction of Cardiac Dysfunction in COVID-19 Patients Using Machine Learning. The COVID-HEART study included adult patients (age >=18 at the time of COVID-19 diagnosis) admitted as inpatients to any of the following hospitals in the Johns Hopkins Health System: Howard County General Hospital, Suburban Hospital, Sibley Memorial Hospital, Johns Hopkins Bayview Medical Center, and Johns Hopkins Hospital. Patient data was collected in the retrospective COVID-19 Precision Medicine Analytics Platform Registry (JH-CROWN). For data from an admission to be included in this study, patients must have had SARS-CoV-2 infection confirmed by polymerase chain reaction (PCR) within 14 days prior to the date of admission or during the admission. The minimum length of time from admission to discharge or death was 4 hours for cardiac arrest prediction and 72 hours for prediction of thromboembolic events, the difference being necessitated by the time granularity with which each outcome could be identified. Data were censored at the time of outcome or discharge.

Additional exclusion criteria were applied for prediction of each outcome separately. Patients were excluded from thromboembolic event prediction if they experienced an imaging-confirmed thromboembolic event or were suspected of experiencing a thromboembolic event immediately prior to admission, which was diagnosed on admission or within 24 hours of admission. For prediction of cardiac arrest, patients were excluded if they experienced cardiac arrest with return of spontaneous circulation immediately prior to admission or if the arrest was precipitated by an event not related to disease severity. For prediction of both outcomes, patients were not excluded based on treatments received, disease severity, need for intensive care, missing clinical variables, or any other reason not listed. Although excluding patients for these reasons may have improved the ML models' performance, this would have resulted in a “clean” cohort not representative of real clinical data, making the risk predictor less useful in a real-world clinical setting. Outcome definition is discussed in Supplementary Methods.

COVID-HEART Predictor Specifications

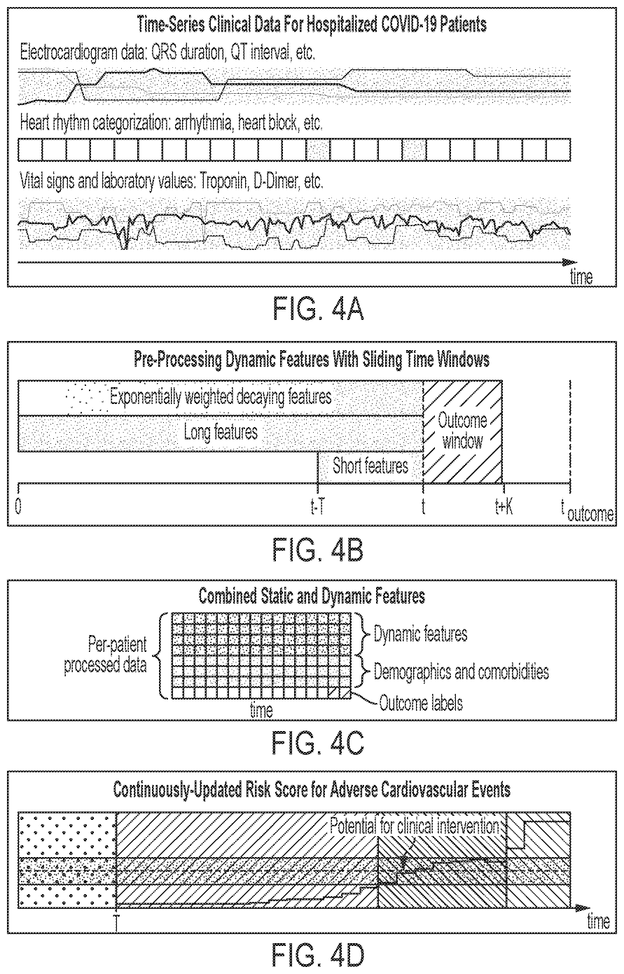

FIG. 4 presents a schematic of the COVID-HEART continuously-updating risk predictor. The TRIPOD guidelines for development, validation, and presentation of a multivariable prediction model were followed here (Supplementary Table 1). The model uses a selection of features extracted from 127 different clinical data inputs (shown schematically in FIG. 4A and presented in detail in Supplmentary Table 2), some of which are associated with CV complications in COVID-19 and in other severe respiratory illnesses. To avoid bias, variables that were directly impacted by a physician's assessment of the patient's condition, such as the fraction of inspired oxygen set on a mechanical ventilator, are excluded. Definition of these predictors, how they were measured, and pre-processing steps undertaken prior to dynamic feature extraction are provided in Supplementary Methods. We intentionally applied minimal “corrections” to the raw clinical data inputs to ensure our development and validation data sets were realistic and that our model could be applied in a real-world clinical setting.

The COVID-HEART predictor was trained to estimate the probability that a patient will experience a particular CV event within a set number of hours (outcome window) after any point during the patient's hospitalization. It used static variables (demographics and comorbidities) and dynamic clinical data collected during time periods of markedly different duration prior to the time point of prediction. Dynamic features were calculated from the processed time-series clinical data inputs as illustrated in FIG. 4B. Each time-point was assigned a binary outcome label indicating whether the patient experienced the outcome of interest in an “outcome window” following the time-point. FIG. 4C schematically shows an array of processed data for a patient who experienced an adverse CV event. The outcome window for prediction of thromboembolic events was 24 hours as this was the minimum interval in which outcomes could be identified. 2 hours was selected as the outcome window for prediction of cardiac arrest based on practical clinical considerations—this would provide healthcare personnel sufficient time for intervention if indicated. Multiple classifier configurations were investigated for prediction of each outcome, detailed in Supplementary Methods.

Classifier Development, Optimization, and Testing

Eligible patients were divided into development and test sets according to the date of their first admission. The cutoff date was selected such that the development set for each outcome included 70% of eligible patients. Patients in the development set for prediction of cardiac arrest were admitted between Mar. 1, 2020 and Nov. 6, 2020; patients in the test set were admitted between Nov. 7, 2020 and Jan. 8, 2020. The cutoff date for prediction of thromboembolic events was Nov. 5, 2020. Data collection ended on the respective cutoff dates for each set.

Classifier development began with five-fold stratified patient-based cross-validation using the development set. We repeated this 20 times for each of the classifier configurations, each time progressively reducing the number of patients used for training and optimization from the full development set by moving the end cutoff date back 1 week (e.g., November 6^{th}, October 30^{th}, October 23^{rd}). At no point did the reduced training set include any patients from the separate test set. Hyperparameters were optimized through cross-validation with a Bayesian hyperparameter search strategy and the optimal classifier configurations were selected based on the aggregated cross-validation area under the receiver operating characteristic curve (AUROC).

Following training and cross-validation of each classifier configuration for prediction of each outcome with the development set, the optimal classifier configuration was trained on the full development set and used to predict the time-series risk of each event for each patient in the respective temporally divided test set. A binary prediction was also made at each time point using the optimal threshold determined by the development data during training. Model performance was assessed by the following metrics: accuracy, balanced accuracy, sensitivity, specificity, and AUROC. As a secondary analysis, the number of time windows predicted positive for patients who eventually experienced events and for patients who did not were compared. Additional analyses to investigate the effects of missing features and the frequency of new clinical data measurements on testing performance were also performed.

Testing was repeated to obtain a 95% confidence interval for each testing performance metric using the final optimized model from each of the 20 iterations of cross-validation. To maintain the temporal nature of the development-test split, we selected an end cutoff date for the test set such that the development and test sets contained 70% and 30% of patients in the reduced data set, respectively. The earliest train-test cutoff date was Jun. 25, 2020; we did not move the train-test cutoff beyond this date to ensure there was enough data to train the predictor. Since there were few events for each outcome, repeating the train-test split in this way provided an accurate estimate of the models' cross-validation performance and performance on a temporally separate test set. All test patient example predictions and data describing the characteristics of the development and testing sets were generated using the model trained with the full development and testing sets (Mar. 1, 2020 to Jan. 8, 2021).

Finally, to assess the predictor's performance when trained and tested with data from patients from different populations, we performed leave-hospital-out validation. This is justified by the fact that each of the five hospitals in the study has different characteristics and serves a different patient population (Supplementary Table 3). Leave-hospital-out validation was performed by removing all patients admitted to one of the five hospitals in the study, repeating the model training and optimization process using data from patients admitted to the remaining four hospitals, and testing the optimized model with data from patients admitted to the left-out hospital. If a patient was transferred between hospitals or had multiple admissions to different hospitals, their admission to the left-out hospital was used in testing and the rest of their data were removed from the training data set.

Results

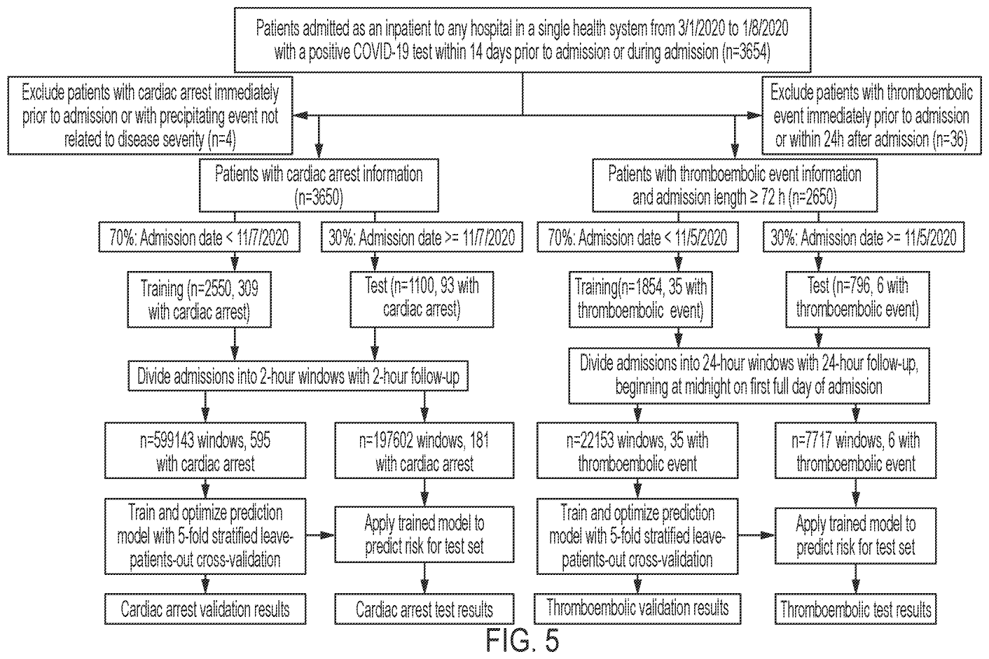

3650 patients met eligibility criteria for prediction of cardiac arrest; 1100 (30.1%) were assigned to the test set according to the date cutoff 2650 patients met eligibility criteria for prediction of thromboembolic events; 796 (30.0%) were assigned to the test set. FIG. 5 shows the flow of patients through the study. Table 1 and Supplementary Table 4 provide demographic and clinical comparisons between patients who did and did not experience each outcome, and between the development and test sets. Overall, 402 out of 3650 patients (11.0%) experienced cardiac arrests, 26 of whom experienced return to spontaneous circulation. Of these, 18 occurred in the intensive care unit (ICU), three occurred in a non-ICU inpatient unit, four occurred in intermediate care/stepdown, and one occurred in long-term inpatient recovery care. 41 out of 2650 (1.5%) eligible patients experienced imaging-confirmed thromboembolic events. 36 additional patients had either an imaging-confirmed thromboembolic event within 24 hours of admission or had clinical suspicion of recent history of thromboembolic events prior to admission and were excluded for those reasons.

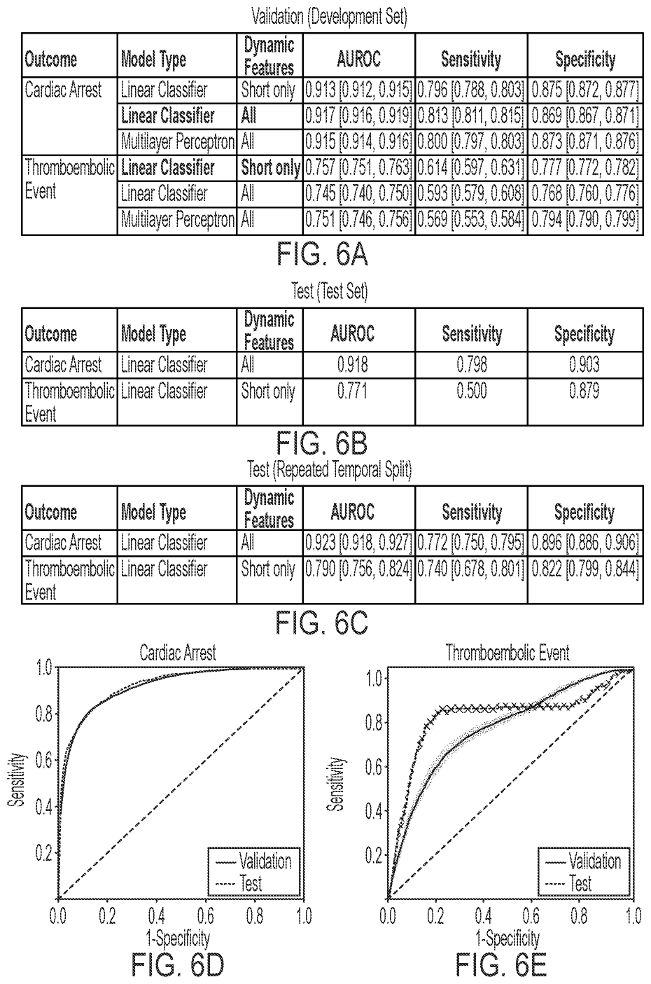

COVID-HEART performance for the two outcomes, in-hospital cardiac arrest and thromboembolic events, is summarized in FIG. 6. Plots of the aggregated cross-validation area under the receiver operating characteristic curves (AUROC) are shown in FIG. 6A. Linear models were optimal for prediction of both outcomes, and included all features for prediction of cardiac arrest and short features only for prediction of thromboembolic events. The optimized COVID-HEART predictor achieved AUROCs of 0.918 and 0.771, sensitivities of 0.768 and 0.500, and specificities of 0.903 and 0.879 for the full test set for prediction of cardiac arrest and thromboembolic events, respectively (FIG. 6B).

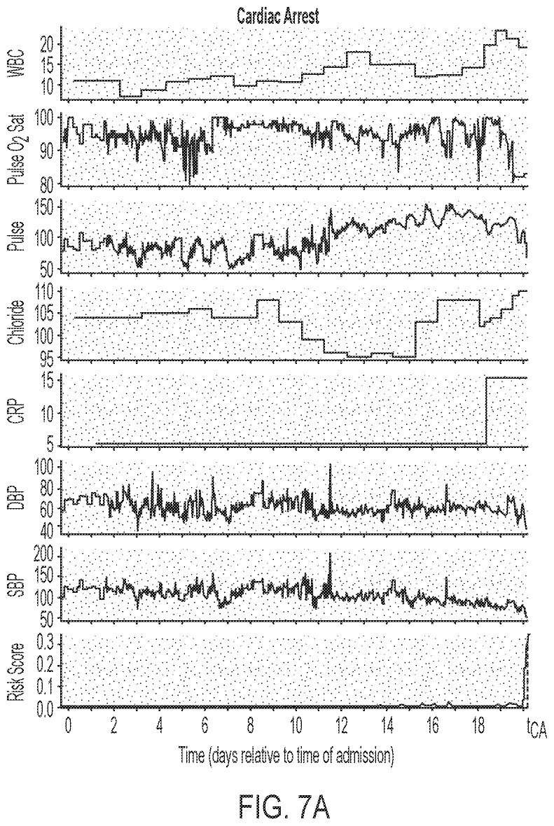

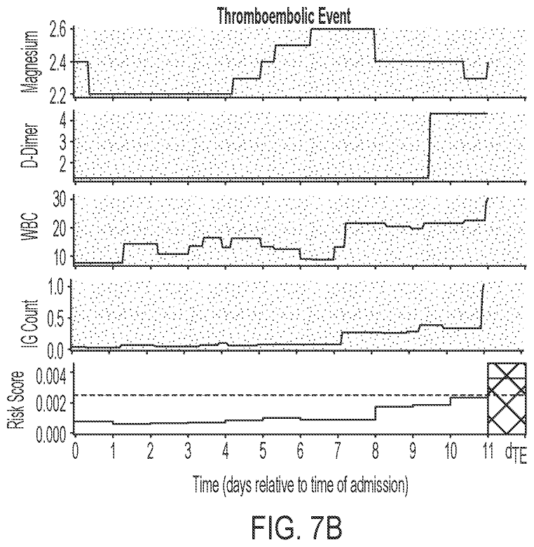

Following the initial development-test split, the results of which are further presented in FIG. 7 and Table 2, the temporal development-test split was repeated and results over 20 iterations were aggregated to obtain 95% confidence intervals for the performance metrics (FIGS. 6C-E). Mean cross-validation and test AUROCs were 0.917 (95% CI: 0.916-0.919) and 0.923 (95% CI: 0.918-0.927) for prediction of cardiac arrest and 0.757 (95% CI: 0.751-0.763) and 0.790 (95% CI: 0.756-0.824) for prediction of thromboembolic events, respectively.

Supplementary Table 5 presents leave-hospital-out cross-validation and testing results. For prediction of cardiac arrest, the mean test AUROC, sensitivity, and specificity for the left-out hospitals were 0.956 (95% CI: 0.936-0.976), 0.885 (95% CI: 0.838-0.933), and 0.887 (95% CI: 0.843-0.932). For prediction of imaging-confirmed thromboembolic events, the mean test AUROC, sensitivity, and specificity for the left-out hospitals were 0.781 (95% CI: 0.642-0.919), 0.453 (95% CI: 0.147-0.760), and 0.863 (95% CI: 0.822-0.904). There were four hospitals in the study at which fewer than 10 imaging-confirmed thromboembolic events were recorded, resulting in a wide confidence interval for sensitivity.

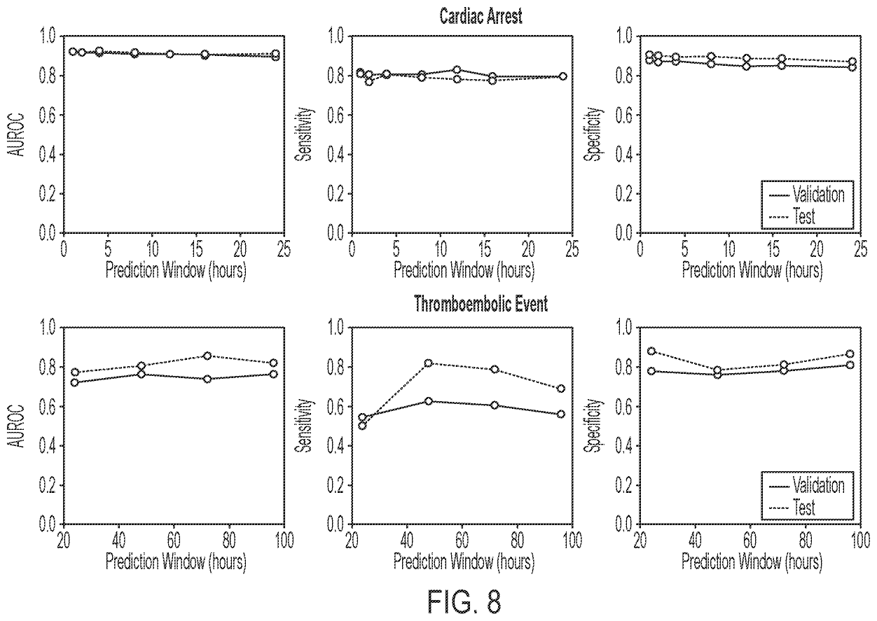

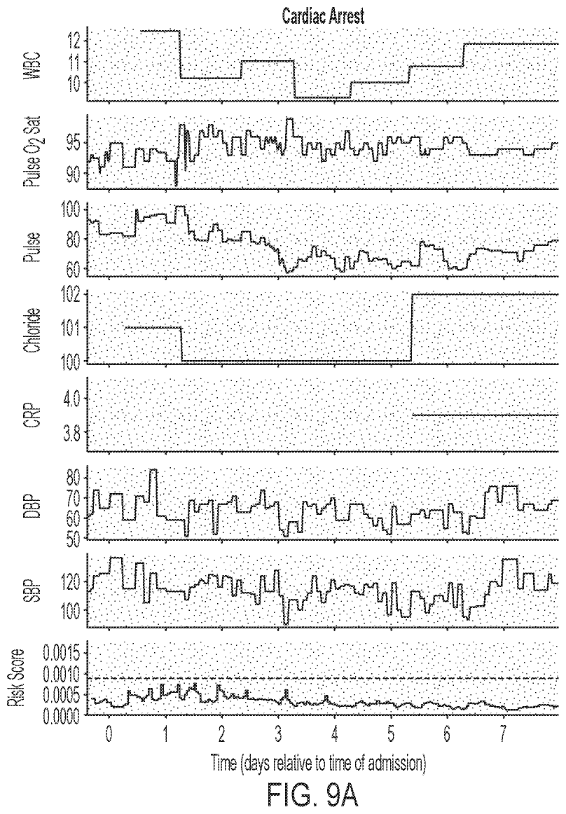

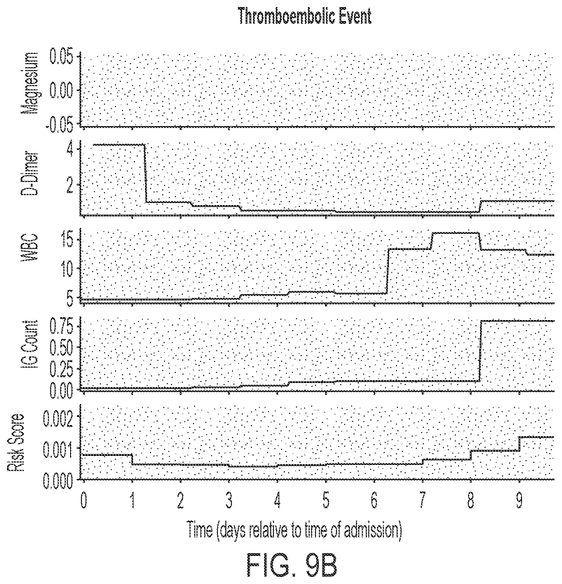

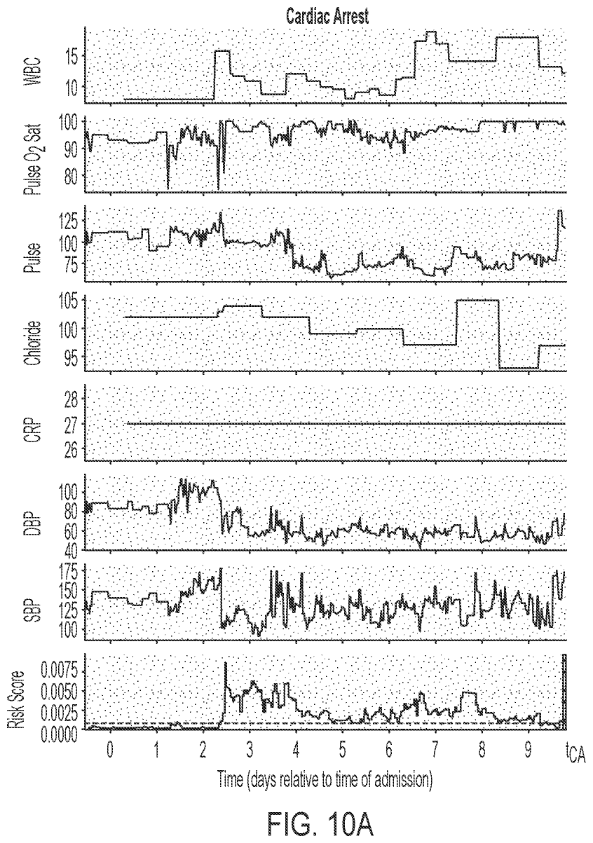

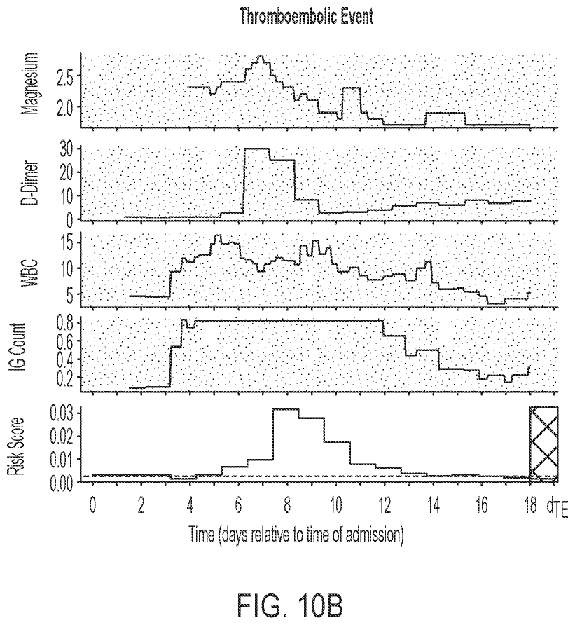

FIG. 8 illustrates the COVID-HEART's capability to accurately predict each CV outcome within outcome windows of different durations. This capability may provide significant clinical value in determining the patient's short-term and longer-term risk, thus ensuring appropriate intervention and resources allocation. As the figures illustrate, cross-validation and test results are comparable, indicating strong generalizability of the COVID-HEART despite statistically significant differences in demographics and prevalence of comorbidities between the development and test sets (Supplementary Table 4). FIG. 4 and FIG. 9 provide examples of time-series clinical data and resulting risk scores for “true positive” and “true negative” predictions for patients in the test set for each CV outcome. FIG. 10 illustrates two incorrect predictions; these are discussed in Supplementary Results.

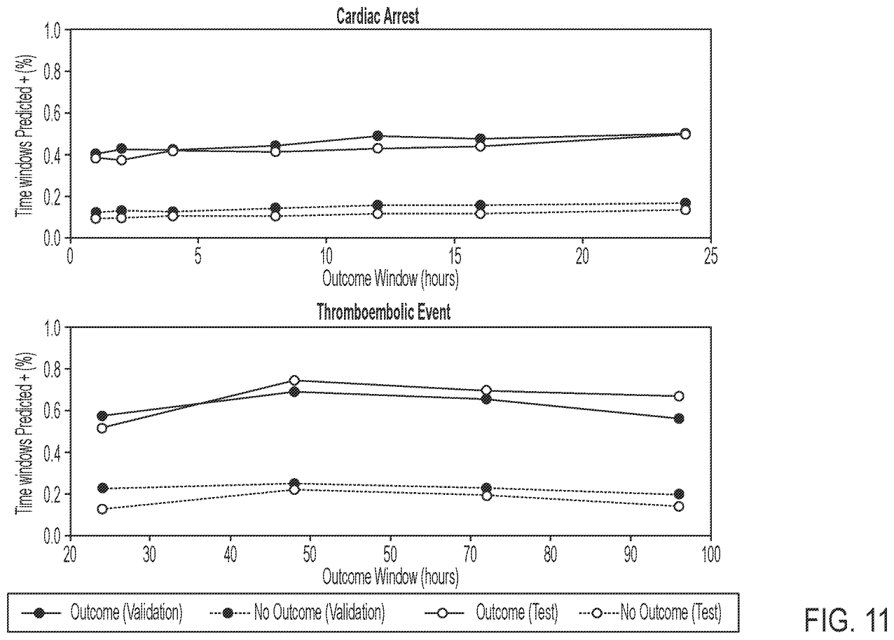

For both outcomes, a larger number of time windows in the test set were predicted positive for patients that eventually experienced the outcome compared to those that did not: 38% vs. 10% for cardiac arrest, 51% vs. 12% for thromboembolic events. The 95% confidence intervals for these measurements over 20 iterations of temporally divided testing were 36%-41% vs. 9%-11% for cardiac arrest and 68%-82% vs. 15%-20% for thromboembolic events. This suggests that the ML model is sensitive in identifying warning signs of an impending adverse event earlier than the pre-specified outcome window (FIG. 11). The interquartile ranges for the median early warning times over 20 iterations of temporally-divided testing were 14-21 hours for cardiac arrest and 12-60 hours for thromboembolic events, although the classifier was trained to predict outcomes within 2 hours for cardiac arrest and 24 hours for thromboembolic events. This could represent a clinically useful “early warning” system.

As it is essential for clinical decision-making to identify the features that most contribute to the predicted risk score for a particular CV outcome, the COVID-HEART predictor was designed to be fully transparent. Table 2 lists up to 20 features with the largest coefficients in the optimal classifier for each of the two CV outcomes. Note that features were normalized prior to classifier training, and that models are not simple logistic regressions, thus interpretation of the coefficients is not straightforward. Many of these features confirm previous observations in cohorts of severely ill COVID-19 patients. For example, lower O₂ saturation is associated with cardiac arrest and multiple coagulation-related labs results are associated with thromboembolic events.

Discussion

In this study, we developed and validated the COVID-HEART predictor, a real-time model that can forecast multiple adverse CV events in hospitalized patients with COVID-19. The COVID-HEART predictor is robust to missing data and can be updated each time new data becomes available, representing a continuously evolving warning system for an impending event. It can also predict the likelihood of an adverse event within multiple timeframes (e.g. 2 hours, 8 hours, 24 hours). Although predictions were made at the same time steps for patients in the test set for consistency with the development set, it is possible to apply the model at any arbitrary time during a patient's hospitalization. We envision that in practice, it could provide the physician with an updated risk score each time any new clinical data input becomes available or only after passing a certain “high risk” threshold, to reduce healthcare provider “alert fatigue”. The COVID-HEART predictor is thus anticipated to be of great clinical use in triaging patients and optimizing resource utilization by identifying at-risk patients in real time. Finally, COVID-HEART is fully transparent thus identifies dynamic predictive features that have not previously been investigated for prediction of these outcomes in patients with COVID-19; these may suggest avenues for future research and personalized targets for clinical intervention.

The COVID-HEART risk prediction approach provides transparency and clinical explainability, including the ability to determine which features are dominant contributors to a patient's risk level at a particular time, which may suggest potential patient-specific targets for clinical intervention. Prediction models for CV adverse events in patients with COVID-19 have been limited by lack of sufficient data, impractical requirements for use (e.g. that all data be available for all patients or that measurements are taken at the same time relative to time of admission), and overly restrictive inclusion/exclusion criteria that result in idealistic training and testing cohorts not representative of real patient data. Our model is designed to handle real-world data, which may include noise, missing variables, and data collected at different points in a patient's hospitalization. The validation and test results indicate strong generalizability despite statistically significant differences between the temporally-divided development and test sets, and between hospitals in the health system. Finally, the inclusion of multiple time-duration features gives the model the “memory” advantages of a long short-term memory neural network without compromising explainability or becoming a “black box”. It is trained in a manner that achieves high sensitivity and specificity despite severe class imbalance. To our knowledge, these techniques have not previously been combined in real-time predictors for CV events.

Models for risk prediction in hospitalized patients have typically focused on predicting mortality risk or length of stay for patients in the ICU. Traditional models incorporate variables thought to indicate physiologic instability or end-organ injury (e.g. respiratory rate, serum bilirubin level, serum creatinine, etc.). While these models generally have good discriminative power, they fail to provide specific, actionable information and simply notify healthcare teams that particular patients are at increased mortality risk at some point in their ICU stay. In most cases, predictive scores are calculated based on the most extreme variable values during the initial 24 hours of the ICU admission, with repeat calculations every 24-72 hours.

Newer models have higher predictive performance compared to traditional models, they are trained to predict the incidence of a particular outcome (e.g. bleeding, renal failure, mortality, etc.) at an indefinite future time. They are not designed to predict the time periods during which patients are at highest risk. Further, in term of ML for risk prediction in COVID-19, prior studies have focused largely on initial diagnosis, mortality, or severity of illness, but none have specifically focused on cardiovascular events, including in-hospital cardiac arrest and thromboembolic events, both clinically important complications with implication for cardiac treatment and monitoring. Moreover, to our knowledge, our model is the first to utilize continuous time series physiologic data as well as laboratory and electrocardiographic data to provide a continuously-updating risk score for an outcome within a particular future time window (e.g. risk of thromboembolic event in the next 24 hours). By providing a risk score for a specific outcome window, our model provides timely, actionable information, allowing the healthcare team to allocate resources and initiate therapies when they are most needed.

With respect to thromboembolic events, we found that 40 out of 41 events occurred in patients already ordered for high-intensity VTE prophylaxis, suggesting an even more aggressive anticoagulant regimen may be needed for those patients identified by the model. Additionally, VTE prophylaxis is one of the treatments most frequently omitted by nursing staff or declined by patients. An analysis of VTE events at our institution over a 72-day period during the Spring 2020 COVID-19 wave demonstrated that 4 out of 11 SARS-CoV-2 positive patients who experienced VTE events had at least one missed dose of VTE prophylaxis. While care providers should ideally strive for 100% compliance with VTE prophylaxis in all eligible patients, the identification of patients at high risk for thromboembolic events may help target these interventions to the patients most in need.

With respect to interventions to address impending cardiac arrest, we found in our detailed chart review that a number of cardiac arrest events were not unprovoked but were a consequence of a precipitating event that altered the patients hemodynamics, such as intubation, patient positioning (e.g. supine to prone), or hemodialysis. Therefore, in addition to predicting unprovoked cardiac arrest (in approximately half of the cases), our model predicted an unstable physiologic state that resulted in cardiac arrest due to otherwise well-tolerated hemodynamic perturbations. Identification of patients as high risk for cardiac arrest would aid clinicians by imploring them to defer any treatments that may provoke cardiac arrest until the patient's physiology recovers. For those treatments that cannot be deferred, identification of high-risk patients would prompt the primary team to assemble specialized staff and equipment, given the high risk of arrest (e.g. calling the anesthesia team for intubation in a high-risk patient, having adequate nursing staff for a possible resuscitation, etc.)

A major barrier to clinical adoption of prognostic machine learning models is the lack of appropriate validation on a representative test cohort. The temporally-divided test sets in this study demonstrated the performance of the predictor on a set of patients admitted after the end of data collection for patients in the development set. A prospective cohort would not be expected to have the same composition as the development set; indeed, there were several statistically significant differences in demographics, clinical characteristics, and prevalence of adverse CV events between the development and tests sets in this study. However, the strong test results show that the predictor is robust to changes in clinical treatment guidelines and evolving demographics. We hypothesize that it maintains its accuracy because it considers data which describe the patient's physiologic state, not variables that are directly influenced by physician input such as ventilator settings or medication use. Further, the predictor maintained strong performance in leave-hospital-out validation, which demonstrated its robustness when trained and tested with data from patients from different populations.

Study Limitations

A limitation in this study is the requirement for imaging confirmation of thromboembolic events. All thromboembolic event diagnoses were adjudicated by a clinician to ensure they were clinically relevant. If the radiologist made an incorrect diagnosis and the adjudicating clinician incorrectly agreed that the event was supported by clinical evidence, this would unfortunately constitute an error in our data set. Similarly, it is likely that patients in the study experienced thromboembolic events that were either the precipitating cause of death or that were not identified on imaging and were therefore not counted as events. There were only 35 patients in the development set with imaging-confirmed thromboembolic events and these outcomes could only be identified per-day, not at the exact time they occurred, as with cardiac arrest. As a result, only a few features could be selected; it is possible that a larger feature set would lead to more accurate prediction of the patients' risk of thromboembolic events since more details of the patients' clinical states could be considered.

Additional limitations stem from the use of the JH-CROWN registry. These include the potential for measurement error, inaccurate patient-reported history (e.g. smoking), and missing data. Another potential limitation is confounding by indication, which means that treatments were selected based on clinical indication. While our model did not include treatments or other variables that were directly influenced by clinical indication, some variables in the model were likely indirectly influenced by clinical indication. For example, the pulse oxygen saturation may have been affected by changes in ventilator settings for patients who were receiving mechanical ventilation. There is also a subgroup of patients who had pre-existing DNR/DNI/comfort care orders. These patients would have received no interventions leading up to an adverse CV event, which means that the sequalae of physiologic changes for these patients may be different than for patients who received interventions prior to an adverse CV event. Finally, there is selection bias inherent to including only patients who sought care at a hospital; patients without insurance, undocumented patients, and patients with other barriers to seeking care may be less likely to be included.

Conclusions

In this study we demonstrated highly accurate prediction of cardiac arrest and thromboembolic events in hospitalized COVID-19 patients using the continuously-updating COVID-HEART predictor. In its current implementation the predictor can facilitate practical, meaningful change in patient triage and the allocation of resources by providing real-time risk scores for CV complications occurring commonly in COVID-19 patients. The COVID-HEART can be re-trained to predict additional adverse CV events including myocardial infarction and arrhythmia. The potential utility of the predictor extends well beyond hospitalized COVID-19 patients, as COVID-HEART could be applied to the prediction of CV adverse events post-hospital discharge or in patients with chronic COVID syndrome (“Long COVID”). Additionally, the ML methodology utilized here could be expanded to use in other clinical scenarios that require screening or early detection, such as risk of hospital readmission, with the goal of improved clinical outcomes through early warnings and resultant opportunity for timely intervention.

Clinical Perspectives

Competency in Practice-Based Learning and Improvement: The COVID-HEART predictor can identify patient at-risk for adverse CV events by quantitatively evaluating changes in dozens of clinical variables, enhancing clinical practice by providing data-driven clinical decision support.

Translation Outlook Implications: Clinical implementation of the algorithm would require a one-time engineering investment to convert the model and pre-processing algorithms into predictive model markup language. The model could then be fully integrated with an electronic health record system and would require no manual input or time investment by a clinician to calculate or view a patient's risk score and the clinical variables that most influenced the score. Prospective validation would be required to increase clinical confidence in the predictor, and a larger training data set would likely improve accuracy of thromboembolic event prediction.

TABLE 1
Characteristics of the entire dataset for each outcome.

| | Cardiac Arrest | | | | | Thromboembolic Event | | | | |
|---|---|---|---|---|---|---|---|---|---|---|
| | Missing | Overall | No Outcome | Outcome | P | Missing | Overall | No Outcome | Outcome | P |
| n | | 3650 | 3248 | 402 | | | 2650 | 2609 | 41 | |

Demographics

| Age (y) | | 0 | 60.9 (18.9) | 59.1 (18.7) | 74.8 (14.1) | <0.001 | 0 | 63.0 (18.1) | 63.0 (18.2) | 64.2 (12.8) | 0.572 |
|---|---|---|---|---|---|---|---|---|---|---|---|
| Sex, n (%) | Male | 0 | 1859 (50.9) | 1639 (50.5) | 220 (54.7) | 0.119 | 0 | 1393 (52.6) | 1364 (52.3) | 29 (70.7) | 0.029 |
| Race, n | Black | 0 | 1281 (35.1) | 1151 (35.4) | 130 (32.3) | <0.001 | 0 | 926 (34.9) | 912 (35.0) | 14 (34.1) | 0.403 |
| (%) | White | | 1309 (35.9) | 1118 (34.4) | 191 (47.5) | | | 984 (37.1) | 972 (37.3) | 12 (29.3) | |
| | Other | | 1060 (29.0) | 979 (30.1) | 81 (20.1) | | | 740 (27.9) | 725 (27.8) | 15 (36.6) | |
| BMI (kg/m2) | | 341 | 30.0 (8.7) | 30.3 (8.8) | 27.4 (7.6) | <0.001 | 231 | 30.0 (8.2) | 30.0 (8.2) | 28.0 (4.7) | 0.006 |
| Number of | 1 | 0 | 3529 (96.7) | 3146 (96.9) | 383 (95.3) | 0.09 | 0 | 2609 (98.5) | 2569 (98.5) | 40 (97.6) | 0.475 |
| admissions, | 2 | | 115 (3.2) | 98 (3.0) | 17 (4.2) | | | 41 (1.5) | 40 (1.5) | 1 (2.4) | |
| n (%) | 3 | | 16 (0.2) | 4 (0.1) | 2 (0.5) | | | | | | |

| | | | | | | | | | | |
|---|---|---|---|---|---|---|---|---|---|---|
| Total length of | 0 | 235.7 (319.2) | 221.7 (305.7) | 349.4 (395.0) | <0.001 | 0 | 291.6 (300.5) | 282.8 (285.0) | 856.8 (593.2) | <0.001 |
| admission(s) (h) | | | | | | | | | | |

Comorbidities

| | | | | | | | | | | |
|---|---|---|---|---|---|---|---|---|---|---|
| Chronic pulmonary | 0 | 1044 (28.6) | 904 (27.8) | 140 (34.8) | 0.004 | 0 | 793 (29.9) | 781 (29.9) | 12 (29.3) | 0.937 |
| disease, n (%) | | | | | | | | | | |
| Diabetes mellitus w/o | 0 | 1418 (38.8) | 1249 (38.5) | 169 (42.0) | 0.181 | 0 | 1130 (42.6) | 1113 (42.7) | 17 (41.5) | 0.996 |
| complications, n (%) | | | | | | | | | | |
| Diabetes mellitus w/ | 0 | 1240 (34.0) | 1067 (32.9) | 173 (43.0) | <0.001 | 0 | 1016 (38.3) | 997 (38.2) | 19 (46.3) | 0.368 |
| complications, n (%) | | | | | | | | | | |
| Valvular disease, n | 0 | 429 (11.8) | 365 (11.2) | 64 (15.9) | 0.008 | 0 | 349 (13.2) | 341 (13.1) | 8 (19.5) | 0.328 |
| (%) | | | | | | | | | | |
| Peripheral vascular | 0 | 586 (16.1) | 487 (15.0) | 99 (24.6) | <0.001 | 0 | 457 (17.2) | 447 (17.1) | 10 (24.4) | 0.311 |
| disorders, n (%) | | | | | | | | | | |
| Pulmonary circulation | 0 | 304 (8.3) | 258 (7.9) | 46 (11.4) | 0.021 | 0 | 229 (8.6) | 209 (8.0) | 20 (48.8) | <0.001 |
| disorders, n (%) | | | | | | | | | | |
| Other neurological | 0 | 1022 (28.0) | 845 (26.0) | 177 (44.0) | <0.001 | 0 | 814 (30.7) | 798 (30.6) | 16 (39.0) | 0.321 |
| disorders, n (%) | | | | | | | | | | |
| Iron deficiency | 0 | 1652 (45.3) | 1412 (43.5) | 240 (59.7) | <0.001 | 0 | 1299 (49.0) | 1270 (48.7) | 29 (70.7) | 0.008 |
| anemia, n (%) | | | | | | | | | | |
| Rheumatoid | 0 | 217 (5.9) | 200 (6.2) | 17 (4.2) | 0.152 | 0 | 167 (6.3) | 165 (6.3) | 2 (4.9) | 1 |
| arthritis/collagen | | | | | | | | | | |
| vascular diseases, n | | | | | | | | | | |
| (%) | | | | | | | | | | |
| Hypertension w/o | 0 | 2466 (67.6) | 2145 (66.0) | 321 (79.9) | <0.001 | 0 | 1874 (70.7) | 1844 (70.7) | 30 (73.2) | 0.861 |
| complications, n (%) | | | | | | | | | | |
| Hypertension w/ | 0 | 1325 (36.3) | 1102 (33.9) | 223 (55.5) | <0.001 | 0 | 1071 (40.4) | 1048 (40.2) | 23 (56.1) | 0.057 |
| complications, n (%) | | | | | | | | | | |
| Obesity, n (%) | 0 | 1384 (37.9) | 1255 (38.6) | 129 (32.1) | 0.012 | 0 | 1051 (39.7) | 1039 (39.8) | 12 (29.3) | 0.226 |
| Coagulopathy, n (%) | 0 | 741 (20.3) | 615 (18.9) | 126 (31.3) | <0.001 | 0 | 606 (22.9) | 589 (22.6) | 17 (41.5) | 0.008 |
| Congestive heart | 0 | 840 (23.0) | 682 (21.0) | 158 (39.3) | <0.001 | 0 | 695 (26.2) | 677 (25.9) | 18 (43.9) | 0.016 |
| failure, n (%) | | | | | | | | | | |
| Fluid and electrolyte | 0 | 2506 (68.7) | 2141 (65.9) | 365 (90.8) | <0.001 | 0 | 1978 (74.6) | 1938 (74.3) | 40 (97.6) | 0.001 |
| disorders, n (%) | | | | | | | | | | |
| Current smoker, n (%) | 0 | 264 (7.2) | 239 (7.4) | 25 (6.2) | 0.084 | 0 | 185 (7.0) | 183 (7.0) | 2 (4.9) | 0.861 |
| History of smoking, n | 0 | 988 (27.1) | 873 (26.9) | 115 (28.6) | 0.137 | 0 | 743 (28.0) | 730 (28.0) | 13 (31.7) | 0.799 |
| (%) | | | | | | | | | | |

Laboratory Tests

| | | | | | | | | | | |
|---|---|---|---|---|---|---|---|---|---|---|
| ProBNP (pg/mL) | 1799 | 2993.7 | 2273.8 | 7360.0 | <0.001 | 1118 | 3139.1 | 3129.2 | 3653.4 | 0.722 |
| | | (8732.2) | (7149.8) | (14392.9) | | | (8890.7) | (8912.9) | (7767.3) | |
| White blood cell | 107 | 7.9 (4.6) | 7.7 (4.4) | 9.5 (5.3) | <0.001 | 41 | 8.0 (4.7) | 8.0 (4.7) | 8.9 (4.4) | 0.193 |
| count (K/uL) | | | | | | | | | | |
| Absolute lymphocyte | 318 | 1.2 (0.9) | 1.2 (0.9) | 1.0 (1.0) | 0.005 | 118 | 1.1 (0.9) | 1.1 (0.9) | 0.9 (0.5) | 0.002 |
| count (K/uL) | | | | | | | | | | |
| D-Dimer (nmol/L) | 608 | 2.4 (5.0) | 2.2 (4.5) | 4.4 (7.5) | <0.001 | 280 | 2.4 (4.9) | 2.4 (4.8) | 6.2 (9.6) | 0.013 |
| Interleukin-6, Serum | 2146 | 376.9 | 153.0 | 2077.8 | 0.096 | 1431 | 237.1 | 230.4 | 485.5 (573.0) | 0.025 |
| (pg/mL) | | (5307.8) | (1032.3) | (15229.1) | | | (1424.2) | (1439.7) | | |
| C-reactive protein | 619 | 26.4 (47.6) | 22.5 (42.5) | 57.1 (69.4) | <0.001 | 281 | 29.3 (49.6) | 29.1 (49.4) | 37.9 (57.0) | 0.338 |
| (mg/L) | | | | | | | | | | |
| Ferritin (mcg/L) | 842 | 985.3 | 924.3 | 1448.4 | <0.001 | 428 | 1043.5 | 1031.9 | 1659.7 | 0.129 |
| | | (1417.5) | (1302.9) | (2035.3) | | | (1406.7) | (1373.3) | (2587.1) | |
| Fibrinogen (mg/dL) | 2485 | 527.9 (175.8) | 527.3 (173.8) | 531.2 (187.2) | 0.797 | 1672 | 535.5 (174.5) | 535.3 (173.6) | 541.0 (206.9) | 0.889 |
| Troponin I (ng/mL) | 2103 | 0.137 (0.778) | 0.086 (0.440) | 0.572 (1.984) | 0.002 | 1430 | 0.148 (0.825) | 0.134 (0.723) | 0.631 (2.470) | 0.25 |

Electrocardiogram Measurements

| | | | | | | | | | | |
|---|---|---|---|---|---|---|---|---|---|---|
| PR interval (ms) | 1033 | 155.5 (28.6) | 155.4 (28.3) | 156.2 (31.1) | 0.694 | 569 | 155.4 (28.6) | 155.4 (28.6) | 153.9 (28.8) | 0.759 |
| QRS duration (ms) | 746 | 92.1 (20.8) | 91.7 (20.3) | 94.7 (23.6) | 0.022 | 331 | 92.4 (21.3) | 92.5 (21.4) | 88.5 (15.0) | 0.102 |
| QT interval (ms) | 747 | 371.6 (49.1) | 371.6 (47.9) | 371.5 (57.0) | 0.977 | 332 | 371.0 (49.2) | 371.0 (49.2) | 374.3 (49.0) | 0.67 |
| QTc interval (ms) | 751 | 447.2 (35.9) | 445.2 (33.9) | 460.7 (45.3) | <0.001 | 336 | 448.2 (36.1) | 448.1 (35.9) | 453.9 (43.3) | 0.402 |
| P axis (degrees) | 1089 | 47.5 (23.3) | 47.2 (23.0) | 49.9 (25.5) | 0.089 | 618 | 46.9 (23.6) | 46.8 (23.7) | 50.7 (14.4) | 0.121 |
| QRS axis (degrees) | 742 | 24.2 (54.6) | 24.9 (53.3) | 19.8 (62.3) | 0.136 | 327 | 22.8 (54.9) | 22.8 (55.1) | 23.7 (46.2) | 0.904 |
| T axis (degrees) | 742 | 44.5 (51.4) | 42.8 (49.2) | 56.2 (63.1) | <0.001 | 327 | 44.9 (51.5) | 44.9 (51.6) | 43.9 (49.7) | 0.889 |
| Ventricular rate | 743 | 90.2 (20.3) | 89.3 (19.5) | 96.8 (24.3) | <0.001 | 328 | 90.8 (19.9) | 90.8 (19.9) | 91.2 (19.2) | 0.889 |
| (bpm) | | | | | | | | | | |
| Atrial rate (bpm) | 788 | 91.2 (23.9) | 90.2 (23.0) | 98.2 (28.7) | <0.001 | 364 | 91.9 (23.7) | 91.9 (23.8) | 91.3 (19.1) | 0.858 |

ECG parameters and lab values are reported as the first result value during the patient's admission. Comorbidities are defined according to diagnosis codes in the Elixhauser comorbidity table. Values are reported as mean (standard deviation) unless otherwise indicated. P-values represent comparison between patients that did and did not experience each outcome and were calculated using the two-sample T-test, Fisher's exact test, or chi-squared test as appropriate. This table was generated using the python package tableone with the Bonferroni correction applied for multiple hypothesis testing.

TABLE 2
Up to 20 features with largest coefficients for prediction of cardiac arrest and thromboembolic events. In the table, ″Feature″ refers to
the processed input to the ML algorithm based on the values of each clinical variable during each time window, and ″Time Duration″ refers to the
length of time over which clinical data values were considered to calculate each feature. Note that features were normalized during pre-processing,
although raw values are shown here, and that values are listed per time-window. These are not the only features included in the classifier for
prediction of cardiac arrest. P-values calculated using two-sample two-sided t-test or chi-squared test as most appropriate. This table was generated
using the python package tableone. Comorbidities, including chronic lung disease and pulmonary circulation disorders, are defined using
ICD-10 codes according to the Elixhauser comorbidity definitions.

| | | | | Train-Cardiac Arrest | | | | Test-Cardiac Arrest | | | |
|---|---|---|---|---|---|---|---|---|---|---|---|
| | | | | Missing | No | | | Missing | No | | |
| Input | Feature | Duration | Coef | (%) | Outcome | Outcome | P | (%) | Outcome | Outcome | P |
| | | | | | 598548 | 595 | | | 197421 | 181 | |
| WBC | Minimum | 2 h | 0.062 | 0.080 | 9.4 (5.0) | 14.5 (7.7) | <0.001 | 0.180 | 9.2 (4.9) | 15.2 (9.0) | <0.001 |
| Age | | | 0.061 | 0.000 | 62.2 (16.7) | 73.9 (14.1) | <0.001 | 0.000 | 66.5 (17.2) | 77.2 (14.1) | <0.001 |
| Pulse O₂ Sat | Maximum | 2 h | −0.057 | 0.100 | 96.5 (2.7) | 91.0 (7.6) | <0.001 | 0.245 | 96.0 (2.7) | 91.3 (7.0) | <0.001 |
| Pulse O₂ Sat | Mean | 2 h | −0.057 | 0.100 | 95.9 (2.8) | 90.2 (7.5) | <0.001 | 0.245 | 95.5 (2.7) | 90.5 (6.9) | <0.001 |
| Pulse | Maximum | 2 h | 0.056 | 0.099 | 87.2 (17.0) | 103.7 (23.4) | <0.001 | 0.243 | 81.4 (17.7) | 102.1 (25.4) | <0.001 |
| Chloride | Minimum | 2 h | 0.057 | 0.072 | 102.5 (5.8) | 104.9 (7.8) | <0.001 | 0.169 | 103.4 (5.8) | 104.7 (8.6) | 0.048 |
| Pulse O₂ Sat | St. Dev. | Long | 0.057 | 0.100 | 2.3 (0.8) | 3.5 (1.4) | <0.001 | 0.245 | 2.2 (0.9) | 3.6 (1.5) | <0.001 |
| CRP | Maximum | 2 h | 0.049 | 0.216 | 24.0 (43.9) | 61.6 (77.4) | <0.001 | 0.348 | 18.1 (35.9) | 37.7 (54.6) | <0.001 |
| Pulse | Mean | 2 h | 0.048 | 0.097 | 85.3 (13.7) | 97.3 (15.8) | <0.001 | 0.240 | 80.0 (14.4) | 96.9 (16.7) | <0.001 |
| DBP | Maximum | 2 h | −0.044 | 0.100 | 70.2 (12.4) | 58.6 (14.8) | <0.001 | 0.244 | 71.1 (12.6) | 60.4 (15.7) | <0.001 |
| SBP | Mean | 2 h | −0.04 | 0.100 | 125.6 | 106.3 (26.0) | <0.001 | 0.244 | 127.8 (19.7) | 109.6 (27.8) | <0.001 |
| | | | | | (19.8) | | | | | | |
| SBP | Minimum | 2 h | −0.04 | 0.100 | 121.7 | 101.2 (26.3) | <0.001 | 0.244 | 124.6 (20.0) | 104.8 (28.0) | <0.001 |
| | | | | | (20.2) | | | | | | |
| Pulse | St. Dev. | 2 h | 0.35 | 0.099 | 10.2 (4.4) | 13.5 (5.1) | <0.001 | 0.243 | 9.7 (4.8) | 15.7 (5.9) | <0.001 |
| Phosphate | Minimum | 2 h | 0.034 | 0.338 | 3.5 (1.1) | 4.3 (1.8) | <0.001 | 0.515 | 3.5 (1.2) | 4.6 (1.9) | <0.001 |
| Anion Gap | Maximum | 2 h | 0.033 | 0.073 | 11.9 (4.1) | 15.4 (6.1) | <0.001 | 0.169 | 10.3 (3.9) | 13.0 (5.9) | <0.001 |
| Weight | | | −0.033 | 0.010 | 2992.3 | 2735.8 | <0.001 | 0.008 | 2933.6 | 2644.5 | <0.001 |
| | | | | | (802.6) | (815.3) | | | (801.4) | (647.2) | |
| Resp. Rate | Mean | Exp. | 0.033 | 0.098 | 22.0 (5.1) | 26.4 (6.1) | <0.001 | 0.241 | 20.8 (4.2) | 24.9 (5.6) | <0.001 |
| | | Decay | | | | | | | | | |
| Resp. Rate | Mean | 2 h | 0.032 | 0.101 | 21.9 (6.1) | 27.0 (8.4) | <0.001 | 0.245 | 20.7 (5.0) | 24.3 (7.9) | <0.001 |
| Platelet Count | Maximum | 2 h | −0.032 | 0.079 | 276.3 | 224.4 | <0.001 | 0.179 | 258.5 | 234.2 | 0.003 |
| | | | | | (136.0) | (127.8) | | | (120.3) | (102.1) | |
| Prothrombin Time | Maximum | 2 h | 0.025 | 0.403 | 12.4 (3.7) | 15.1 (7.1) | <0.001 | 0.676 | 12.4 (3.8) | 12.9 (3.3) | 0.104 |
| | | | | Train-Thromboembolic Event | | | | Test-Thromboembolic Event | | | |
| | | | | Missing | No | | | Missing | No | | |
| Input | Feature | Duration | Coef | (%) | Outcome | Outcome | P | (%) | Outcome | Outcome | P |
| | | | | | 22118 | 35 | | | 7711 | 6 | |
| Pulm. Circ. | | Static | 0.329 | 0.000 | 0.1 (0.3) | 0.5 (0.5) | <0.001 | 0.000 | 0.1 (0.3) | 0.7 (0.5) | 0.046 |
| Magnesium | Maximum | 24 h | 0.231 | 0.247 | 2.2 (0.4) | 2.6 (0.6) | 0.004 | 0.431 | 2.2 (0.4) | 2.1 (0.4) | 0.594 |
| D-Dimer | Maximum | 24 h | 0.200 | 0.211 | 4.6 (7.4) | 10.6 (10.4) | 0.003 | 0.330 | 3.1 (5.6) | 7.3 (6.7) | 0.299 |
| WBC | Mean | 24 h | 0.088 | 0.096 | 9.4 (4.9) | 13.8 (7.3) | 0.002 | 0.210 | 9.5 (4.9) | 9.3 (7.9) | 0.949 |
| WBC | Minimum | 24 h | 0.086 | 0.096 | 8.7 (4.5) | 12.7 (6.8) | 0.002 | 0.210 | 8.7 (4.6) | 8.5 (7.5) | 0.956 |
| IG Count | Mean | 24 h | 0.077 | 0.189 | 0.2 (0.2) | 0.4 (0.5) | 0.010 | 0.315 | 0.2 (0.2) | 0.2 (0.1) | 0.500 |
| WBC | Maximum | 24 h | 0.067 | 0.096 | 10.2 (5.4) | 14.8 (7.4) | 0.001 | 0.210 | 10.3 (5.4) | 11.5 (10.9) | 0.822 |
| IG Count | Minimum | 24 h | 0.056 | 0.189 | 0.2 (0.2) | 0.4 (0.4) | 0.012 | 0.315 | 0.1 (0.2) | 0.2 (0.1) | 0.616 |
| IG Count | Maximum | 24 h | 0.043 | 0.189 | 0.2 (0.3) | 0.4 (0.5) | 0.010 | 0.315 | 0.2 (0.3) | 0.4 (0.4) | 0.372 |

Abbreviations: white blood cell count (WBC), pulse oxygen saturation (pulse O2 sat), c-reactive protein (CRP), diastolic blood pressure (DBP), systolic blood pressure (SBP), respiratory rate (resp. rate), pulmonary circulation disorders (Pulm. Circ.), immature granulocyte (IG), exponentially weighted decaying (exp. decay), coefficient (coef).

Supplementary Methods

Patient Population

The JH-CROWN COVID-19 registry includes patients of all ages seen, since Jan. 1, 2020, at any Johns Hopkins Medical Institution facility (inpatient, outpatient, in-person, video consult, or lab order) with confirmed COVID-19 or suspected of having COVID-19. The cohort is defined as having a completed laboratory test for COVID-19 (whether positive or negative), having an ICD-10 diagnosis of COVID-19 (recorded at the time of encounter, entered on the problem list, entered as medical history, or appearing as a billing diagnosis), or flagged as a “patient under investigation” for suspected or confirmed COVID-19 infection. Further details are available on the Johns Hopkins Institute for Clinical and Translational Research website.

Additional inclusion and exclusion criteria were applied for the COVID-HEART study, which resulted in a subset of the JH-CROWN registry being included. FIG. 2 illustrates the flow of patients through the study. The COVID-HEART study included adult patients (age >=18 at the time of COVID-19 diagnosis) admitted as inpatients to any of the following hospitals in the Johns Hopkins Health System: Howard County General Hospital, Suburban Hospital, Sibley Memorial Hospital, Johns Hopkins Bayview Medical Center, and Johns Hopkins Hospital. For an admission to be included, patients must have had a laboratory-confirmed SARS-CoV-2 infection within 14 days prior to the date of admission or during the admission. The minimum length of time from admission to discharge or death was 4 hours for cardiac arrest prediction and 72 hours for prediction of thromboembolic events, the difference being necessitated by the time granularity with which each outcome could be identified, discussed in further detail in the following section. Time spent in the emergency department did not count towards the admission duration, but if a patient had clinical data (e.g. laboratory values or vital signs) recorded in the emergency department prior to admission, those values were used to initialize the clinical data inputs at the start of their inpatient admission. Data were censored at the time of outcome or discharge.

Multiple admissions were handled as follows. If a patient was transferred between hospitals in the health system and thus had two admissions recorded in the JH-CROWN registry with a gap of fewer than 4 hours, it was treated as a single admission. However, if a patient was discharged and re-admitted to the same hospital or a different hospital more than 4 hours later, the admissions were treated separately, and all dynamic clinical data inputs were “reset” for the second admission. Admission-based inclusion/exclusion criteria were applied separately for each admission.

Outcome Definition

The primary outcome for each patient was whether they experienced in-hospital cardiac arrest and/or an imaging-confirmed thromboembolic event.

In-hospital cardiac arrest included all-cause mortality and cardiac arrest with return of spontaneous circulation. All-cause mortality was defined according to the time of death recorded in the JH-CROWN database. Cardiac arrest with return of spontaneous circulation was defined as documentation in the medical record of a non-perfusing rhythm and subsequent initiation of chest compressions and other resuscitative measures by the health care team. All cardiac arrest events were considered, regardless of the influence of any precipitating events such as patient position change or respiratory decompensation. These were queried by searching for the ICD-10 code ‘I46.X’ within the problem list and encounter diagnosis list. We performed chart review to adjudicate all ICD-10-based cardiac arrest diagnoses according to the above definition. For patients with multiple cardiac arrests, the first outcome was used, and the remainder of their data were censored.

Thromboembolic outcomes included pulmonary embolism confirmed on computed tomography (CT) angiography of the chest, non-hemorrhagic stroke confirmed on CT of the head, and deep venous thrombosis confirmed on either vascular ultrasound or CT of the abdomen or pelvis. Findings that were diagnosed or clinically apparent on initial presentation (confirmed on imaging within 24 hours of presentation) were excluded from analysis. For a patient with multiple adverse coagulation outcomes during their hospitalization, the first outcome was used. We note that such a strict outcome definition could mean that some outcomes were missed, especially if a patient's immediate cause of death was a thromboembolic event or if the event was confirmed by point-of-care ultrasound that was not recorded in the imaging procedure list. However, we found that alternative outcome definition methods (such as ICD-10 diagnosis codes) resulted in many “false positive” outcomes upon chart review, so this method was chosen to ensure all thromboembolic events were confirmed with a consistent, objective level of clinical certainty.

Predictors

Supplementary Table 2 lists all clinical data inputs from which predictors were extracted. Here, we discuss the definition of these predictors, how they were measured, and pre-processing steps undertaken prior to dynamic feature extraction.

Demographic inputs included age, gender, weight, height, body mass index, and race. Gender was defined as the patient's legal gender (Male or Female) as listed in the electronic health record (EHR). Race was self-reported and divided into three categories according to the most common values in the JH-CROWN registry: Black, white, and other. The inclusion of race in machine learning models is controversial. However, there is significant evidence that Black patients and other patients of color experience worse outcomes in COVID-19. We were concerned that by not including race, our model may fail to account for a higher baseline risk of adverse outcomes among Black patients in the study cohort's geographic area. Future work, prior to a prospective study, could include a re-analysis of the current results to ensure that the predictions are not systematically less accurate for any demographic group. Comorbidities were defined by mapping ICD-10 codes according to the Elixhauser comorbidity definitions using the hcuppy python library.

Vital signs were extracted from flowsheet data recorded in the EHR and added to the JH-CROWN registry. Pulse measurements were excluded if the recording was 0. Both systolic blood pressure (SBP) and diastolic blood pressure (DBP) were recorded using either a blood pressure cuff or an arterial line. These were combined into a single input. If a given time point had measurements for SBP and DBP with both modalities, the arterial line measurement took priority. SBP measurements between 30 and 270 mmHg were considered valid. DBP measurements between 30 and 130 mmHg were considered valid. If the difference between SBP and DBP was less than 15 mmHg, both measurements were considered invalid. Respiratory rates between 4 and 52 breaths per minute were considered valid. Temperatures between 89° F. and 105° F. (31.7° C.-40.6° C.) were considered valid. Pulse oxygen saturation between 30% and 100% was considered valid. Other flowsheet data, such as fraction of inspired oxygen and positive end expiratory pressure, were not included as these are directly influenced by a physician's assessment of the patient's condition, rather than physiologic data reflecting the patient's condition in an unbiased manner. Heart rhythm indicators were also extracted from flowsheet data.

Laboratory tests results were extracted from EHR data and were time-stamped at the time the result was received, not the time of collection. This was done to ensure the model was trained with realistic data; in a prospective study it would not be possible to know the result of a laboratory test for a patient at the time the specimen would be collected.

ECG measurements were extracted from the 12-lead ECG. As with laboratory tests, these measurements were time-stamped at the time the result was received, not the time of the procedure. Parameters (QRS duration, QT interval, etc.) were evaluated by the clinician who interpreted the ECG results.

For all clinical data inputs, outliers that were >5 standard deviations from the mean were removed. This threshold was chosen to avoid excluding abnormal but non-erroneous values. We intentionally applied minimal “corrections” to clinical data inputs to ensure our development and validation data sets were realistic and that our model could be applied in a real-world clinical setting.

The testing data set was identified and sequestered from the training data prior to model development. Since this was a retrospective study and did not include any data collected prospectively, there was no need of blind assessment of predictors for patients in the testing set. Patients were assigned to development and test sets after predictors were collected and outcomes were defined.

Sample Size

The study size was determined by the number of patients in the JH-CROWN registry who met all inclusion and exclusion criteria for prediction of each outcome.

Feature Extraction and Missing Data

Here we present methods for extracting features from dynamic clinical data and handling of missing predictors in the analysis. All pre-processing steps were performed using the Python Pandas data analysis library. Laboratory tests, vital signs, and ECG measurements were handled similarly. For each patient, each measurement for each variable within these categories was associated with a time-stamp at which the measurement was received. Data were re-sampled in 30-minute increments for the prediction of cardiac arrest and in 1-hour increments for the prediction of thromboembolic events with mean interpolation if multiple measurements were made in a window. Missing values from the beginning of the patient's hospitalization (e.g., if they did not have a measurement for a particular laboratory test until hour 48, or at any point during their hospitalization) were left empty and handled later, within the modeling pipeline. Missing values following a measurement (e.g., if a patient had an ECG at hour 12, then did not have another ECG until hour 48) were handled with forward filling; each variable was held constant until a new measurement was made.

In the remainder of the Methods, we refer to “time point”, “time window”, “feature window”, “outcome window”, and “positive” time window. A time point indicates a single moment in time. The time window before a time point, during which clinical data are collected and features are extracted, is referred to as the “feature window”. The time window immediately after, in which the risk of a particular CV outcome is predicted, is referred to as the “outcome window”. “Positive time windows” or “positive time points” are time windows or points for which the patient experienced the CV outcome of interest in the following outcome window.

Following the preprocessing steps described above, dynamic features were calculated from the processed time-series clinical data inputs as illustrated in FIG. 1B. “Short features” encompassed a short window of time immediately preceding the time point at which the prediction was to be made. For example, if the feature window length was 2 hours, these features would include the mean, standard deviation, minimum, maximum, and amplitude of first frequency in Fourier space of the variable over the preceding 2 hours. “Long features” included the mean, standard deviation, minimum, and maximum over the patient's entire hospitalization preceding the time point at which the prediction was to be made. “Exponentially weighted decay features” also encompassed the patient's entire hospitalization preceding the time point at which the prediction was to be made, but the measurements were exponentially weighted according to how recently they were made with more recent measurements weighted more strongly and a half-life of 1 day.

Heart rhythm indicators were re-sampled similarly to other dynamic clinical data inputs but were treated discretely. For each window, two variables were recorded for each heart rhythm indicator (Atrial fibrillation, heart block, etc.): a binary indicator of whether the patient experienced that heart rhythm within the window and an integer-valued variable indicating how many times that heart rhythm was noted within the window. It was assumed that if a patient did not have any heart rhythm annotations within a particular hour, they did not experience an abnormal heart rhythm during that window, so missing values were filled in with zero for both the binary indicator variable and integer-valued variable. “Short features” and “long features” were calculated for each heart rhythm indicator but included only the sum (total number of times each was recorded over the interval) and maximum (maximum number of times a rhythm was recorded in a single hour within the interval).

Dynamic features were extracted at each time point during each patient's hospitalization. The time-step between time points at which predictions were made was 1 hour for prediction of cardiac arrest and 24 hours for prediction of thromboembolic events. For thromboembolic events, each time window began at midnight; for cardiac arrest, each time window began at the top of the hour, commencing with the first full hour after the patient was admitted as an inpatient. The difference in time-step was due to the difference in the time granularity of the outcome labels. Although cardiac arrest outcomes could be defined by the minute in which they occurred, and thus it would be appropriate to use a time-step as small as 1 minute, 1 hour was chosen to balance computational costs with the desire to train the classifier with as much data as possible. A time-step of 1 hour resulted in 599143 time windows for the development set, which produced an accurate, generalizable classifier as demonstrated by the strong cross-validation and testing results for prediction of cardiac arrest.

Statistical Analysis Methods

Two linear and one non-linear classifier configurations were investigated for prediction of each outcome using the feature windows and outcome windows described above (both 2-hour windows for cardiac arrest and 24-hour windows for thromboembolic events): a linear classifier with short features only, a linear classifier with all feature types, and a non-linear multi-layer perceptron classifier with all feature types. Here, we discuss the specifications for each model. Unless otherwise stated, methods were the same for all three classifier configurations.

Model Specification

The models evaluated included a linear classifier trained with stochastic gradient descent and a multi-layer perceptron model. The linear model was chosen as it is highly explainable (not a “black box”), it is efficient to train with hundreds of thousands of time windows, and it can be updated without requiring full re-training. The learning rate of the linear model was set to “optimal” with early stopping and balanced class weight. The multi-layer perceptron model is similarly efficient to train and can be updated without full re-training. Although it is more difficult to interpret, we chose to include it to assess whether a non-linear model could better represent the relationships between clinical data inputs. As COVID-19 treatment paradigms change, we expect that model updating would be necessary to retain accuracy among evolving clinical practices.

Pre-processing steps included removal of features which were missing for >60% of time windows, mean-value imputation for numerical features that were missing (typically at the beginning of a patient's hospitalization or if a certain laboratory test was never performed for a given patient), scaling all numerical features to zero mean and unit variance. Finally, feature selection was incorporated using a lasso regression model for sparsity. This feature selection method was chosen as it is not biased towards selecting high-cardinality variables over variables with fewer discrete values (e.g., binary comorbidity features), in contrast with other popular feature selection methods such as the random forest algorithm. We used ANOVA F-value-based feature selection for prediction of thromboembolic events and significantly restricted the number of features that could be selected to reduce the likelihood of over-fitting due to the very small number of events in the development set.

Five-fold stratified group cross-validation was used to optimize hyperparameters of the COVID-HEART predictor. Groups were assigned such that all time points from each patient were held out in the same fold of cross-validation. Hyperparameters were optimized for all steps in the pipeline with 150 iterations of Bayesian optimization using the python package scikit-optimize for prediction of thromboembolism (since the time step was 24 hours, there were fewer time windows and thus training was more efficient) and 50 iterations for prediction of cardiac arrest to maximize the validation AUROC. Convergence of AUROC was visualized to confirm that the number of iterations was appropriate for each outcome.

Hyperparameters for the linear model included the maximum number of features selected, the loss function (hinge, log, modified Huber, Huber, squared hinge), the regularization penalty (L1, L2, and L1L2), the regularization strength, and the L1 ratio for L1L2 regularization. Losses were weighted during training to strongly penalize errors for positive time windows. If the optimal loss function of the linear classifier was not log or modified Huber, the optimized classifier was calibrated after training to provide risk probabilities in addition to binary predictions. Hyperparameters for the multi-layer perceptron classifier included the maximum number of features selected, the number and size of hidden layers, the regularization strength, the learning rate decay schedule (constant, inverse scaling, or adaptive), and the initial learning rate.

Model Testing

Following design of feature extraction methods, model development, and model training, the optimal models for prediction of each outcome were re-fit using the entire development set and calibrated if necessary. Static and dynamic features were then calculated for patients in the testing set using the same methods as for the development set. The fitted models were used to predict the risk of each CV outcome at each time point for each patient in the testing set. A binary prediction was also made at each time point using the optimal threshold determined by the development data during training. Models were tested using repeated temporal validation and leave-hospital-out validation.

Comparison with Clinical Metrics

To benchmark the COVID-HEART predictor's performance against current clinical guidelines, we assessed each patient's venous thromboembolism (VTE) risk according to the risk tiers in use at the Johns Hopkins Health System. These guidelines recommended that all COVID-19 intensive care unit (ICU) patients were considered high risk and received high-intensity VTE prophylaxis. Additional high-risk factors included pregnancy, active malignancy, history of prior VTE, sickle cell disease, known thrombophilia, and D-Dimer >1.5 mg/L at any time during the patient's hospitalization. All other patients were considered lower risk and received standard VTE prophylaxis.

Model Updating

The temporally divided testing set was sequestered until the end of model development. There were no changes made to the model following testing. After determining the optimal classifier configuration for prediction of each event within the outcome windows specified above, we performed a secondary analysis in which we varied the length of the outcome window to investigate whether the COVID-HEART predictor could forecast outcomes within multiple intervals. At this point, the feature extraction and modeling methodology was pre-determined and only the outcome window was varied.

Development Vs. Validation

All patients were from a subset of the JH-CROWN registry. There were no differences between development and test data in setting, outcome, and predictors. The eligible dates of admission were different between the development and test sets. If a patient had multiple COVID-19-related admissions, they were assigned to either the development or test set according to their earliest admission date.

Supplementary Results

Participants

FIG. 5 shows the flow of participants through the study. 3650 patients met eligibility criteria for cardiac arrest prediction. 402 experienced cardiac arrest. Table 1 provides a clinical and demographic comparison of patients who did and did not experience cardiac arrest. Overall, patients who experienced cardiac arrest were older (mean age 74.8 years vs. 59.1 years, p<0.001) and spent more time in the hospital (349.4 hours vs. 221.7 hours, p<0.001). They were more likely to have a history of chronic pulmonary disease (34.8% vs. 27.8%, p=0.004), diabetes mellitus with complications (43.0% vs. 32.9%, p<0.001), valvular disease (15.9% vs. 11.2%, p=0.008), peripheral vascular disorders (24.6% vs. 15.0%, p<0.001), pulmonary circulation disorders (11.4% vs. 7.9%), neurological disorders (44.0% vs. 26.0%, p<0.001), iron deficiency anemia (59.7% vs. 43.5%, p<0.001), hypertension with and without complications (55.5% vs. 33.9%, p<0.001 and 79.9% vs. 66.0%, p<0.001, respectively), coagulopathy (31.3% vs. 18.9%, p<0.001), congestive heart failure (39.3% vs. 21.0%, p<0.001), and fluid and electrolyte disorder (90.8% vs. 65.9%, p<0.001). They were less likely to be obese (32.1% vs. 38.6%, p=0.012), but this may reflect loss of body mass associated with old age.

In investigating their first laboratory measurements on admission to the hospital for a select subset of laboratory tests that have been shown to be associated with adverse outcomes in COVID-19, patients who experienced cardiac arrest had statistically significantly higher NT-pro-brain natriuretic peptide (pro-BNP), white blood cell count, D-Dimer, C-reactive protein, ferritin, and troponin. They had statistically significantly lower absolute lymphocyte count. Of note, many patients were missing measurements for several of these tests. Finally, patients who experienced cardiac arrest had statistically significantly longer QRS duration, longer QTc interval, greater T axis, higher ventricular rate, and higher atrial rate on their first ECG after admission to the hospital.

These patients were divided into development and test sets using a cutoff date so that all data were collected for patients in the development set before any patients in the test set were admitted for the first time. The cutoff date was chosen so that 30% of the total data set was assigned to the test set. This resulted in a development set of 2550 patients in which 309 (12.1%) experienced cardiac arrest and a testing set of 1100 patients in which 93 (8.5%) experienced cardiac arrest. We hypothesize that the statistically significant (p=0.001) difference in outcome prevalence can be attributed to rapidly changing treatment paradigms in response to the developing understanding of the disease over the first year of the pandemic. Supplementary Table 4 provides a comparison between the development and testing sets. Since patients in the test set were admitted after the last date of data collection for patients in the development set, there were several statistically significant differences in demographics, comorbidities, laboratory tests, and ECG measurements between the two sets of patients. This is advantageous as it allows us to demonstrate the COVID-HEART predictor's performance on a realistic test set, considering rapidly evolving treatment guidelines and changing demographics as virus spread rises and falls among different communities; a prospective study would also be limited to patients admitted after the final date of data collection for patients in the development set. 2686 patients met eligibility criteria for thromboembolic event prediction. 36 of these patients were excluded for having an thromboembolic event within 24 hours of admission, this usually indicated that the event occurred prior to admission and was confirmed with imaging on admission. 41 of the remaining 2650 patients experienced imaging-confirmed in-hospital thromboembolic events. Table 1 provides a clinical and demographic comparison of patients who did and did not experience thromboembolic events. Patients who experienced thromboembolic events had longer admission duration (856.8 hours vs. 282.8 hours, p<0.001). They were more likely to have pulmonary circulation disorders (48.8% vs. 8.0%, p<0.001), iron deficiency anemia (70.7% vs. 48.7%, p=0.008), coagulopathy (41.5% vs. 22.6%, p=0.008), congestive heart failure (43.9% vs. 25.9%, p=0.016), and fluid and electrolyte disorders (97.6% vs. 74.3%, p=0.001). On admission, they had statistically significantly lower absolute lymphocyte count and statistically significantly higher D-dimer, and IL-6.

These patients were also temporally divided into a development set of 1854 patients in which 35 (1.9%) experienced imaging-confirmed thromboembolic events and a testing set of 796 patients in which 6 (0.8%) experienced imaging-confirmed thromboembolic events. Supplementary Table 4 provides a comparison between the development and testing sets; there are several statistically significant differences in clinical and demographic characteristics. As with the development and test sets for prediction of cardiac arrest, these differences likely reflect evolving treatment guidelines and changing demographics over the first year of the pandemic.

Table 1 indicates the number of patients for which each measurement was missing. This does not necessarily mean they never had a measurement for a certain variable. It may mean that they had a recording at a hospital in a different health system prior to being transferred to a hospital in the Johns Hopkins Health System or that data was missing from the JH-CROWN registry. This is an inherent limitation in the use of retrospective registry data, discussed in further detail in Supplementary Methods.

Model Specification

The optimal model for prediction of cardiac arrest with a feature window of 2 hours, outcome window of 2 hours, and time step of 1 hour was a linear model with features selected from short, long, and exponentially weighted decaying features. The optimal hyperparameters included 61 features selected, Huber loss, L2 regularization penalty, epsilon (determines threshold at which it becomes less important to get the prediction exactly correct) of 0.009, and regularization strength of 0.029. The optimal model for prediction of thromboembolic outcomes with a feature window of 24 hours, outcome window of 24 hours, and time step of 24 hours was a linear model with short features only. The optimal hyperparameters included 9 features selected, log loss, L2 regularization penalty, and regularization strength of 0.307.

Table 2 lists the features with largest absolute coefficients in the model for prediction of each outcome along with their values for time windows in the development and test sets. Feature selection was performed using the development set only. The most important features for prediction of cardiac arrest within 2 hours included age, many vital signs, and lab tests that indicate inflammation, cardiac function, and metabolic function. Several of these have previously been noted as predictors of various adverse outcomes in COVID-19. This serves as a “sanity check” that the model is learning reasonable associations between predictors and outcomes, despite its novel real-time nature.

The features with largest absolute coefficients for prediction of thromboembolic events within 24 hours were derived from D-dimer, magnesium, white blood cell count, immature granulocytes, and pulmonary circulation disorders. Other variables were also associated with thromboembolic events (Table 1), but only a few features could be included in the model due to the small number of events in the development set. D-dimer suggests the presence of blood clots being degraded by fibrinolysis and is associated with thromboembolic events. Magnesium promotes fibrinolysis and may be given as an anti-coagulant, so the features extracted from magnesium measurements may indirectly reflect physician assessment that the patient is at high risk for thromboembolic events. Finally, white blood cell count is often elevated in patients with pulmonary embolism and deep vein thrombosis, which explains why it is predictive of thromboembolic events.

Model Performance

The overall performance of the optimal model for prediction of each outcome is discussed in the main text results. Here, we discuss the results in more detail, including patient-specific example predictions for patients in the test set for each outcome and leave-hospital-out validation and testing results.

Test Patient Example Predictions

The first example predictions are the “true positive” predictions for one patient in the test set for each outcome, as shown in FIG. 4. In predicting the risk of cardiac arrest within 2 hours for the patient whose data is shown in FIG. 4A, results show that their risk is very low for the first 17 days of their hospitalization as their white blood cell count trends upward and vital signs fluctuate. Over the last 2 days of their hospitalization, their pulse oxygen saturation and pulse decrease, while their chloride and white blood cell count increase. The effects of these changes are reflected in the patient's risk score, which increases very rapidly in the 18 hours leading up to the time at which the patient experienced cardiac arrest. Although there are concerning fluctuations in vital signs prior to the day on which the patient experienced cardiac arrest, including multiple drops in pulse oxygen saturation to 80%, the COVID-HEART predictor is successful in determining when the patient becomes at risk of cardiac arrest in the short-term by considering these and other inputs together.

In predicting the risk of a thromboembolic event within 1 day for the patient whose data is shown in FIG. 4B, the results demonstrate that the risk score is low for the first 11 days of the patient's hospitalization, then it crosses the binary risk threshold immediately prior to the 12^{th} day when the patient experienced an imaging-confirmed thromboembolic event. The patient's white blood cell count is high, which may raise clinical suspicion for infection. However, the patient does not have remarkably elevated D-dimer or other traditional risk markers for a thromboembolic event. This highlights the usefulness of the COVID-HEART risk predictor in identifying at-risk patients that may not have raised clinical suspicion for an impending thromboembolic event.

FIG. 9 shows an example of a “true negative” prediction for one patient in the test set for each outcome. The cardiac arrest risk score for the patient whose data is shown in FIG. 9A remains below 0.08% for their entire hospitalization. This patient has several drops pulse oxygen saturation below 90% and isolated drops in systolic and diastolic blood pressures, but the COVID-HEART risk predictor successfully assessed their risk as low. This patient did not experience cardiac arrest and was discharged after 8 days in the hospital. The thromboembolic event risk score for the patient whose data is shown in FIG. 9B remains near 0.1% and below the binary risk threshold for their entire hospitalization. This patient did not experience any imaging-confirmed thromboembolic events and was discharged after 9 days. This also illustrates the COVID-HEART risk predictor's ability to cope with missing clinical data; the patient has no recorded measurements for magnesium during their hospitalization.

FIG. 10 shows an example of an incorrect prediction for one patient in the test set of each outcome. The patient whose clinical data is shown in FIG. 10A experienced cardiac arrest on day 10 of their hospitalization. Their risk score for cardiac arrest increased rapidly at the end of the 3^{rd} day, corresponding to drops in pulse oxygen saturation below 80%, an increase in their pulse, and increase in their white blood cell count. However, it then decreased and continued fluctuating, remaining mostly above the binary risk threshold, until the time at which they experienced cardiac arrest. Although they were not at the highest risk immediately before they experienced cardiac arrest, their risk score was still above the binary threshold immediately prior to the event. Thus, although this was technically a correct prediction, we focus on the risk score spike on day 3 and the elevated risk score between days 3-10 as examples of false positive predictions.

The patient whose clinical data is shown in FIG. 10B experienced an imaging-confirmed thromboembolic event on the 19^{th} day of their hospitalization. Their risk score was low for the first 5 days of their hospitalization, then rose sharply in response to significantly elevated D-dimer. It is possible that the patient experienced a thromboembolic event at this time, but they did not have a thromboembolic event confirmed by imaging until the 19^{th} day, 12 days later. It is also possible that the patient was treated with anti-coagulation therapy in response to the elevated D-dimer, which could have prevented a thromboembolic event during the D-dimer spike. This example highlights the need for further investigation of incorrect predictions and a prospective study of the COVID-HEART predictor in a larger cohort, which would make it possible to identify the timing of thromboembolic events more precisely.

Predicting CV Events Within Various Outcome Windows

After determining the optimal classifier configuration for prediction of each outcome with pre-determined outcome windows and short feature windows, we performed a series of experiments in which we varied the duration of the outcome window and repeated the training, optimization, and validation process with the full development and test sets as described in Methods. FIG. 8 shows the results of varying the outcome window for prediction of each outcome. For prediction of cardiac arrest, the outcome window can vary from 1 to 24 hours with little change in AUROC, sensitivity, and specificity. This analysis shows that the COVID-HEART predictor can predict cardiac arrest within multiple outcome window durations, representing a continuous early warning system for cardiac arrest that may be able to determine both the patient's short-term and longer-term risk. FIG. 8 also presents numerical results for all outcome windows for the prediction of thromboembolic events. When the feature window is held constant at 24 hours, the results are similar for prediction of thromboembolic events within 1, 2, 3, and 4 days.

Performance of Current Clinical Risk Prediction Methods

41 patients in the study experienced imaging-confirmed thromboembolic events. Of these, 40 patients were considered high-risk by the Johns Hopkins VTE risk guidelines and were on high-intensity prophylaxis at the time of the event. 2609 patients did not experience imaging confirmed thromboembolic events, yet 2046 of these patients met high-risk criteria. The Johns Hopkins VTE risk guidelines achieved a sensitivity of 0.976, specificity of 0.216, positive predictive value of 0.019, and negative predictive value of 0.998.

Supplementary Tables

| | Section/Topic | | | |
|---|---|---|---|---|
| Title and abstract | Checklist Item | | | Page |
| Title | 1 | D; | Identify the study as developing and/or validating a | 1 |
| | | [symbol] | multivariable prediction model, the target population, and | |
| | | | the outcome to be predicted. | |
| Abstract | 2 | D; | Provide a summary of objectives, study design, setting, | 2 |
| | | [symbol] | participants, sample size, predictors, outcome, statistical | |
| | | | analysis, results, and conclusions. | |
| Background and | 3a | D; | Explain the medical context (including whether | 4-5 |
| objectives | | [symbol] | diagnostic or prognostic) and rationale for developing or | |
| | | | validating the multivariable prediction model, including | |
| | | | references to existing models. | |
| | 3b | D; | Specify the objectives, including whether the study | 4-5 |
| | | [symbol] | describes the development or validation of the model or | |
| | | | both. | |
| Source of data | 4a | D; | Describe the study design or source of data (e.g., | 5-6 |
| | | [symbol] | randomized trial, cohort, or registry data), separately for | |
| | | | the development and validation data sets, if applicable. | |
| | 4b | D; | Specify the key study dates, including start of accrual; | 8 |
| | | [symbol] | end of accrual; and, if applicable, end of follow-up. | |
| Participants | 5a | D; | Specify key elements of the study setting (e.g., primary | 5-6 |
| | | [symbol] | care, secondary care, general population) including | |
| | | | number and location of centres. | |
| | 5b | D; | Describe eligibility criteria for participants. | 5-6 |
| | | [symbol] | | |
| | 5c | D; | Give details of treatments received, if relevant. | N/A |
| | | [symbol] | | |
| Outcome | 6a | D; | Clearly define the outcome that is predicted by the | 35-36 |
| | | [symbol] | prediction model, including how and when assessed. | |
| | 6b | D; | Report any actions to blind assessment of the outcome to | 38 |
| | | [symbol] | be predicted. | |
| Predictors | 7a | D; | Clearly define all predictors used in developing or | 36-38, |
| | | [symbol] | validating the multivariable prediction model, including | 63 |
| | | | how and when they were measured. | |
| | 7b | D; | Report any actions to blind assessment of predictors for | 38 |
| | | [symbol] | the outcome and other predictors. | |
| Sample size | 8 | D; | Explain how the study size was arrived at. | 38 |
| | | [symbol] | | |
| Missing data | 9 | D; | Describe how missing data were handled (e.g., complete- | 39 |
| | | [symbol] | case analysis, single imputation, multiple imputation) | |
| | | | with details of any imputation method. | |
| Statistical | 10a | D | Describe how predictors were handled in the analyses. | 39-40 |
| analysis | 10 | D | Specify type of model, all model-building procedures | 41-42 |
| methods | [symbol] | | (including any predictor selection), and method for | |
| | | | internal validation. | |
| | 10c | V | For validation, describe how the predictions were | 42-43 |
| | | | calculated. | |
| | 10 | D; | Specify all measures used to assess model performance | 8 |
| | [symbol] | [symbol] | and, if relevant, to compare multiple models. | |
| | 10e | V | Describe any model updating (e.g., recalibration) arising | N/A |
| | | | from the validation, if done. | |
| Risk groups | 11 | D; | Provide details on how risk groups were created, if done. | N/A |
| | | [symbol] | | |
| Development vs. | 12 | V | For validation, identify any differences from the | 44-47 |
| validation | | | development data in setting, eligibility criteria, outcome, | |
| | | | and predictors. | |
| Participants | 13a | D; | Describe the flow of participants through the study, | 26 |
| | | [symbol] | including the number of participants with and without the | |
| | | | outcome and, if applicable, a summary of the follow-up | |
| | | | time. A diagram may be helpful. | |
| | 13 | D; | Describe the characteristics of the participants (basic | |
| | [symbol] | [symbol] | demographics, clinical features, available predictors), | 23-25 |
| | | | including the number of participants with missing data | |
| | | | for predictors and outcome. | |
| | 13c | V | For validation, show a comparison with the development | 66-88 |
| | | | data of the distribution of important variables | |
| | | | (demographics, predictors and outcome). | |
| Model development | 14a | D | Specify the number of participants and outcome events in | 10 |
| | | | each analysis. | |
| | 14 | D | If done, report the unadjusted association between each | |
| | [symbol] | | candidate predictor and outcome. | |
| Model specification | 15a | D | Present the full prediction model to allow predictions for | 32-33 |
| | | | individuals (i.e., all regression coefficients, and model | |
| | | | intercept or baseline survival at a given time point). | |
| | 15 | D | Explain how to the use the prediction model. | 43 |
| | [symbol] | | | |
| Model performance | 16 | D; | Report performance measures (with CIs) for the | 10-12 |
| | | [symbol] | prediction model. | |
| Model-updating | 17 | V | If done, report the results from any model updating (i.e., | |
| | | | model specification, model performance). | |
| Limitations | 18 | D; | Discuss any limitations of the study (such as | 16-17 |
| | | [symbol] | nonrepresentative sample, few events per predictor, | |
| | | | missing data). | |
| Interpretation | 19a | V | For validation, discuss the results with reference to | 13-16 |
| | | | performance in the development data, and any other | |
| | | | validation data. | |
| | 19 | D; | Give an overall interpretation of the results, considering | 13-16 |
| | [symbol] | [symbol] | objectives, limitations, results from similar studies, and | |
| | | | other relevant evidence. | |
| Implications | 20 | D; | Discuss the potential clinical use of the model and | 13-16 |
| | | [symbol] | implications for future research. | |
| Supplementary | 21 | D; | Provide information about the availability of | 19-20 |
| information | | [symbol] | supplementary resources, such as study protocol, Web | |
| | | | calculator, and data sets. | |
| Funding | 22 | D; | Give the source of funding and the role of the funders for | 1 |
| | | [symbol] | the present study. | |

[symbol]

indicates data missing or illegible when filed

| Category | Clinical Data Inputs |
|---|---|
| Demographics (6) | Age, gender, weight, height, body mass index, race |
| Comorbidities (30) | Current smoker, history of smoking, chronic pulmonary disease, |
| | diabetes mellitus with complications, diabetes mellitus without |
| | complications, lymphoma, valvular disease, psychosis, peripheral |
| | vascular disorder, pulmonary circulation disorders, hypothyroidism, |
| | alcohol abuse, neurological disorders, deficiency anemia, renal failure, |
| | liver disease, rheumatoid arthritis/collagen, solid tumor without |
| | metastasis, metastatic cancer, drug abuse, depression, HIV/AIDS, |
| | hypertension with complications, hypertension without complications, |
| | obesity, coagulopathy, peptic ulcer disease, congestive heart failure, |
| | paralysis, fluid and electrolyte disorders |
| Vital signs (6) | Pulse, systolic blood pressure, diastolic blood pressure, respiratory rate, |
| | temperature, pulse oxygen saturation |
| Lab tests (60) | NT-pro-brain natriuretic peptide, white blood cell count, absolute |
| | lymphocyte count, D-dimer, nucleated red blood cell count, |
| | lymphocytes %, polymorphonuclear, neutrophils %, absolute neutrophil |
| | count, basophil %, eosinophil %, monocyte %, atypical lymphocytes, |
| | eosinophil number, heparin (anti-Xa), interleukin-6 (IL-6) serum, C- |
| | reactive protein, ferritin, fibrinogen, troponin I, troponin T, bands, |
| | creatinine serum, blood urea nitrogen (BUN), BUN/creatinine ratio, |
| | sodium, potassium, alkaline phosphatase, hematocrit, phosphate, |
| | hemoglobin, mean corpuscular hemoglobin, red blood cell count, urine |
| | red blood cell count, red blood cell distribution width, immature |
| | granulocyte %, immature granulocyte number, glomerular filtration rate, |
| | glucose, chloride, carbon dioxide, total protein, calcium, procalcitonin, |
| | magnesium, alanine aminotransferase, aspartate aminotransferase, |
| | AST/ALT ratio, bilirubin, lactate whole blood, lactate dehydrogenase, |
| | erythrocyte sedimentation rate, lactate dehydrogenase, platelet count, |
| | mean platelet volume, activated partial thromboplastin time (APTT), |
| | APTT ratio, prothrombin time, anion gap, mean corpuscular volume, |
| | nitrite |
| ECG (9) | PR interval, QRS duration, QT interval, QTc interval, P axis, QRS axis, |
| | T axis, ventricular rate, atrial rate |
| Heart rhythm | Atrial fibrillation, atrial flutter, sinus arrhythmia, ectopy, heart block, |
| indicators (16) | sinus tachycardia, sinus bradycardia, junctional rhythm, ventricular |
| | tachycardia, asystole, ventricular paced rhythm, normal sinus rhythm, |
| | atrial paced rhythm, A-V sequential paced rhythm, supraventricular |
| | tachycardia, agonal |

Supplementary Table 2. Clinical data inputs from which features were derived. These are discussed in further detail in Methods. Comorbidities are defined using ICD-10 codes according to the Elixhauser comorbidity definitions.

| | | | | COVID-19 | COVID-19 |
|---|---|---|---|---|---|
| Hospital | Beds | Setting | Location | Patients (TE) | Patients (CA) |
| Johns Hopkins | 1162 | Urban | Baltimore, MD | 879 (28 events = | 1213 (98 events = |
| Hospital | | | | 3.2%) | 8.1%) |
| Bayview | 463 | Urban | Baltimore, MD | 494 (6 events = | 792 (38 events = |
| Medical | | | | 1.2%) | 4.8%) |
| Center | | | | | |
| Howard | 243 | Suburban | Columbia, MD | 541 (1 event = | 831 (69 events = |
| County | | | | 0.2%) | 8.3%) |
| General | | | | | |
| Hospital | | | | | |
| Sibley | 288 | Urban | Washington, | 225 (1 event = | 314 (36 events = |
| Memorial | | | DC | 0.4%) | 11.5%) |
| Hospital | | | | | |
| Suburban | 228 | Suburban | Bethesda, MD | 581 (5 events = | 768 (156 events = |
| Hospital | | | | 1.2%) | 20.3%) |

Supplementary Table 3. Characteristics of five hospitals to which patients in the study were admitted. Patient counts indicate the number of patients with a valid inpatient admission at each hospital—an admission with a transfer between hospitals is counted here as a separate admission to each of the hospitals, provided the patient's time at each hospital meets inclusion criteria with respect to duration and proximity to a positive COVID-19 test.

| | Cardiac Arrest | | | | | Thromboembolic Event | | | | |
|---|---|---|---|---|---|---|---|---|---|---|
| | Missing | Overall | Development | Validation | P | Missing | Overall | Development | Validation | P |
| n | | 3650 | 2550 | 1100 | | | 2650 | 1854 | 796 | |

Demographics

| Age (y) | | 0 | 60.9 (18.9) | 59.5 (18.7) | 64.0 (18.9) | <0.001 | 0 | 63.0 (18.1) | 61.7 (18.0) | 66.2 (18.0) | <0.001 |
|---|---|---|---|---|---|---|---|---|---|---|---|
| Sex, n (%) | Male | 0 | 1859 (50.9) | 1311 (51.4) | 548 (49.8) | 0.397 | 0 | 1393 (52.6) | 983 (53.0) | 410 (51.5) | 0.501 |
| Race, n (%) | Black | 0 | 1281 (35.1) | 939 (36.8) | 342 (31.1) | <0.001 | 0 | 926 (34.9) | 692 (37.3) | 234 (29.4) | <0.001 |
| | White | | 1309 (35.9) | 787 (30.9) | 522 (47.5) | | | 984 (37.1) | 604 (32.6) | 380 (47.7) | |
| | Other | | 1060 (29.0) | 824 (32.3) | 236 (21.5) | | | 740 (27.9) | 558 (30.1) | 182 (22.9) | |
| BMI (kg/m2) | | 341 | 30.0 (8.7) | 30.2 (8.9) | 29.7 (8.2) | 0.125 | 223 | 30.0 (8.2) | 30.1 (8.2) | 29.6 (8.2) | 0.19 |
| Number of | 1 | 0 | 3529 (96.7) | 2475 (97.1) | 1054 (95.8) | 0.018 | 0 | 2609 (98.5) | 1829 (98.7) | 780 (98.0) | 0.274 |
| admissions, n | 2 | | 115 (3.2) | 69 (2.7) | 46 (4.2) | | | 41 (1.5) | 25 (1.3) | 16 (2.0) | |
| (%) | 3 | | 6 (0.2) | 6 (0.2) | | | | | | | |

| | | | | | | | | | | |
|---|---|---|---|---|---|---|---|---|---|---|
| Total length of | 0 | 235.7 | 251.3 | 199.5 | <0.001 | 0 | 291.6 | 307.1 | 255.7 | <0.001 |
| admission(s) (h) | | (319.2) | (348.6) | (233.5) | | | (300.5) | (317.3) | (253.7) | |
| Experienced outcome | 0 | 402 (11.0) | 309 (12.1) | 93 (8.5) | 0.001 | 0 | 41 (1.5) | 35 (1.9) | 6 (0.8) | 0.046 |

Comorbidities

| | | | | | | | | | | |
|---|---|---|---|---|---|---|---|---|---|---|
| Chronic pulmonary | 0 | 1044 (28.6) | 680 (26.7) | 364 (33.1) | <0.001 | 0 | 793 (29.9) | 519 (28.0) | 274 (34.4) | 0.001 |
| disease, n (%) | | | | | | | | | | |
| Diabetes mellitus w/o | 0 | 1418 (38.8) | 987 (38.7) | 431 (39.2) | 0.815 | 0 | 1130 (42.6) | 795 (42.9) | 335 (42.1) | 0.737 |
| complications, n (%) | | | | | | | | | | |
| Diabetes mellitus w/ | 0 | 1240 (34.0) | 837 (32.8) | 403 (36.6) | 0.028 | 0 | 1016 (38.3) | 695 (37.5) | 321 (40.3) | 0.182 |
| complications, n (%) | | | | | | | | | | |
| Valvular disease, n | 0 | 429 (11.8) | 266 (10.4) | 163 (14.8) | <0.001 | 0 | 349 (13.2) | 221 (11.9) | 128 (16.1) | 0.005 |
| (%) | | | | | | | | | | |
| Peripheral vascular | 0 | 586 (16.1) | 365 (14.3) | 221 (20.1) | <0.001 | 0 | 457 (17.2) | 291 (15.7) | 166 (20.9) | 0.002 |
| disorders, n (%) | | | | | | | | | | |
| Pulmonary | 0 | 304 (8.3) | 202 (7.9) | 102 (9.3) | 0.197 | 0 | 229 (8.6) | 158 (8.5) | 71 (8.9) | 0.796 |
| circulation disorders, | | | | | | | | | | |
| n (%) | | | | | | | | | | |
| Other neurological | 0 | 1022 (28.0) | 696 (27.3) | 326 (29.6) | 0.16 | 0 | 814 (30.7) | 564 (30.4) | 250 (31.4) | 0.647 |
| disorders, n (%) | | | | | | | | | | |
| Iron deficiency | 0 | 1652 (45.3) | 1146 (44.9) | 506 (46.0) | 0.58 | 0 | 1299 (49.0) | 915 (49.4) | 384 (48.2) | 0.63 |
| anemia, n (%) | | | | | | | | | | |
| Rheumatoid | 0 | 217 (5.9) | 144 (5.6) | 73 (6.6) | 0.279 | 0 | 167 (6.3) | 108 (5.8) | 59 (7.4) | 0.146 |
| arthritis/collagen | | | | | | | | | | |
| vascular diseases, n | | | | | | | | | | |
| (%) | | | | | | | | | | |
| Hypertension w/o | 0 | 2466 (67.6) | 1676 (65.7) | 790 (71.8) | <0.001 | 0 | 1874 (70.7) | 1272 (68.6) | 602 (75.6) | <0.001 |
| complications, n (%) | | | | | | | | | | |
| Hypertension w/ | 0 | 1325 (36.3) | 881 (34.5) | 444 (40.4) | 0.001 | 0 | 1071 (40.4) | 718 (38.7) | 353 (44.3) | 0.008 |
| complications, n (%) | | | | | | | | | | |
| Obesity, n (%) | 0 | 1384 (37.9) | 972 (38.1) | 412 (37.5) | 0.733 | 0 | 1051 (39.7) | 742 (40.0) | 309 (38.8) | 0.591 |
| Coagulopathy, n (%) | 0 | 741 (20.3) | 522 (20.5) | 219 (19.9) | 0.732 | 0 | 606 (22.9) | 427 (23.0) | 179 (22.5) | 0.799 |
| Congestive heart | 0 | 840 (23.0) | 543 (21.3) | 297 (27.0) | <0.001 | 0 | 695 (26.2) | 455 (24.5) | 240 (30.2) | 0.003 |
| failure, n (%) | | | | | | | | | | |
| Fluid and electrolyte | 0 | 2506 (68.7) | 1753 (68.7) | 753 (68.5) | 0.893 | 0 | 1978 (74.6) | 1397 (75.4) | 581 (73.0) | 0.218 |
| disorders, n (%) | | | | | | | | | | |
| Current smoker, n | 0 | 264 (7.2) | 180 (7.1) | 84 (7.6) | 0.734 | 0 | 185 (7.0) | 126 (6.8) | 59 (7.4) | 0.264 |
| (%) | | | | | | | | | | |
| History of smoking, n | 0 | 988 (27.1) | 627 (24.6) | 361 (32.8) | <0.001 | 0 | 743 (28.0) | 473 (25.5) | 270 (33.9) | <0.001 |
| (%) | | | | | | | | | | |

Laboratory Tests

| | | | | | | | | | | |
|---|---|---|---|---|---|---|---|---|---|---|
| ProBNP (pg/mL) | 1799 | 2993.7 | 2843.1 | 3357.4 | 0.246 | 1118 | 3139.1 | 2894.4 | 3754.2 | 0.089 |
| | | (8732.2) | (8768.1) | (8642.1) | | | (8890.7) | (8870.8) | (8921.0) | |
| White blood cell | 107 | 7.9 (4.6) | 8.0 (4.7) | 7.6 (4.2) | 0.01 | 41 | 8.0 (4.7) | 8.1 (4.9) | 7.7 (4.2) | 0.016 |
| count (K/uL) | | | | | | | | | | |
| Absolute lymphocyte | 318 | 1.2 (0.9) | 1.2 (0.9) | 1.1 (0.9) | 0.005 | 118 | 1.1 (0.9) | 1.1 (0.9) | 1.0 (0.7) | <0.001 |
| count (K/uL) | | | | | | | | | | |
| D-Dimer (nmol/L) | 608 | 2.4 (5.0) | 2.4 (4.9) | 2.5 (5.1) | 0.376 | 280 | 2.4 (4.9) | 2.4 (4.9) | 2.5 (5.0) | 0.556 |
| Interleukin-6, Serum | 2146 | 376.9 | 470.0 | 109.1 | 0.058 | 1431 | 237.1 | 274.0 | 128.8 | 0.067 |
| (pg/mL) | | (5307.8) | (6133.6) | (961.9) | | | (1424.2) | (1523.8) | (1075.4) | |
| C-reactive protein | 619 | 26.4 (47.6) | 28.6 (49.8) | 21.0 (41.1) | <0.001 | 281 | 29.3 (49.6) | 31.7 (52.2) | 23.3 (41.8) | <0.001 |
| (mg/L) | | | | | | | | | | |
| Ferritin (mcg/L) | 842 | 985.3 | 997.7 | 952.1 | 0.454 | 428 | 1043.5 | 1065.2 | 985.1 | 0.222 |
| | | (1417.5) | (1407.0) | (1445.7) | | | (1406.7) | (1427.0) | (1349.9) | |
| Fibrinogen (mg/dL) | 2485 | 527.9 | 528.6 | 520.9 | 0.696 | 1672 | 535.5 | 536.9 | 521.9 | 0.456 |
| | | (175.8) | (173.7) | (196.8) | | | (174.5) | (173.1) | (187.4) | |
| Troponin I (ng/ml) | 2103 | 0.137 | 0.136 | 0.137 | 0.985 | 1430 | 0.148 | 0.153 | 0.137 | 0.759 |
| | | (0.778) | (0.730) | (0.885) | | | (0.825) | (0.809) | (0.865) | |

Electrocardiogram Measurements

| | | | | | | | | | | |
|---|---|---|---|---|---|---|---|---|---|---|
| PR interval (ms) | 1033 | 155.5 (28.6) | 154.9 (28.7) | 157.0 | 0.092 | 569 | 155.4 (28.6) | 154.7 (28.7) | 157.2 (28.3) | 0.072 |
| | | | | (28.4) | | | | | | |
| QRS duration (ms) | 746 | 92.1 (20.8) | 91.5 (19.7) | 93.6 (23.2) | 0.02 | 331 | 92.4 (21.3) | 91.7 (20.1) | 94.4 (24.1) | 0.01 |
| QT interval (ms) | 747 | 371.6 (49.1) | 369.6 (48.0) | 376.5 | 0.001 | 332 | 371.0 (49.2) | 368.3 (47.6) | 377.7 (52.4) | <0.001 |
| | | | | (51.5) | | | | | | |
| QTc interval (ms) | 751 | 447.2 (35.9) | 446.6 (36.4) | 448.7 | 0.147 | 336 | 448.2 (36.1) | 447.3 (36.4) | 450.5 (35.0) | 0.05 |
| | | | | (34.7) | | | | | | |
| P axis (degrees) | 1089 | 47.5 (23.3) | 47.3 (23.1) | 48.1 (23.8) | 0.413 | 618 | 46.9 (23.6) | 46.6 (23.5) | 47.6 (23.8) | 0.41 |
| QRS axis (degrees) | 742 | 24.2 (54.6) | 23.8 (53.7) | 25.3 (56.8) | 0.51 | 327 | 22.8 (54.9) | 23.0 (53.8) | 22.5 (57.8) | 0.876 |
| T axis (degrees) | 742 | 44.5 (51.4) | 44.0 (49.6) | 45.8 (55.5) | 0.418 | 327 | 44.9 (51.5) | 44.6 (50.2) | 45.7 (54.7) | 0.655 |
| Ventricular rate | 743 | 90.2 (20.3) | 90.9 (20.1) | 88.6 (20.9) | 0.007 | 328 | 90.8 (19.9) | 91.6 (19.5) | 88.7 (20.5) | 0.001 |
| (bpm) | | | | | | | | | | |
| Atrial rate (bpm) | 788 | 91.2 (23.9) | 91.7 (23.3) | 90.1 (25.4) | 0.117 | 364 | 91.9 (23.7) | 92.7 (23.3) | 89.9 (24.6) | 0.014 |

Supplementary Table 4. Characteristics of the training and test sets for each outcome. ECG parameters and lab values are reported as the first result value during the patient's admission. Comorbidities are defined according to diagnosis codes in the Elixhauser comorbidity table. Values are reported as mean (standard deviation) unless otherwise indicated. P-values represent comparison between patients in the training and test sets for each outcome and were calculated using the two-sample T-test, Fisher's exact test, or chi-squared test as appropriate. This table was generated using the python package tableone with the Bonferroni correction applied for multiple hypothesis testing.

| | Events | AUROC | | Sensitivity | | Specificity | |
|---|---|---|---|---|---|---|---|
| Left-Out Hospital | Test | Validation | Test | Validation | Test | Validation | Test |
| | | Cardiac Arrest | | | | | |
| Johns Hopkins | 98 | 0.964 | 0.914 | 0.889 | 0.842 | 0.890 | 0.833 |
| Hospital | | | | | | | |
| Bayview Medical | 38 | 0.965 | 0.975 | 0.873 | 0.803 | 0.907 | 0.971 |
| Center | | | | | | | |
| Howard County | 69 | 0.967 | 0.972 | 0.890 | 0.912 | 0.900 | 0.905 |
| General Hospital | | | | | | | |
| Sibley Memorial | 36 | 0.968 | 0.970 | 0.916 | 0.944 | 0.903 | 0.890 |
| Hospital | | | | | | | |
| Suburban Hospital | 156 | 0.970 | 0.950 | 0.867 | 0.926 | 0.915 | 0.838 |
| | | Thromboembolic Event | | | | | |
| Johns Hopkins | 28 | 0.879 | 0.729 | 0.917 | 0.500 | 0.747 | 0.848 |
| Hospital | | | | | | | |
| Bayview Medical | 6 | 0.789 | 0.794 | 0.625 | 0.167 | 0.838 | 0.912 |
| Center | | | | | | | |
| Howard County | 1 | 0.753 | 0.963 | 0.686 | 1.000 | 0.723 | 0.784 |
| General Hospital | | | | | | | |
| Sibley Memorial | 1 | 0.739 | 0.511 | 0.594 | 0.000 | 0.816 | 0.863 |
| Hospital | | | | | | | |
| Suburban Hospital | 5 | 0.792 | 0.906 | 0.625 | 0.600 | 0.800 | 0.908 |

Supplementary Table 5. Leave-hospital-out cross-validation and testing results. Each row contains cross-validation results when patients who were admitted to that hospital at any time during the study are left out of the development set, and testing results for patients admitted to that hospital using the model trained and optimized with the development set. If a patient has a valid admission at multiple hospitals, data from their admission to the left-out hospital is assigned to the test set and their other admissions are excluded from the development set to prevent data leakage.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1.-3. (canceled)

4. A method of generating a model for prognosing a cardiovascular (CV) outcome for a monitored subject infected with severe acute respiratory syndrome coronavirus-2 (SARS-CoV-2) at least partially using a computer, the method comprising:
generating, by the computer, a first set of data values of a first plurality of dynamic clinical parameters associated with at least a first plurality of monitored reference subjects infected with the SARS-CoV-2, wherein at least a subset of the first set of data values comprises one or more time-series data values;
processing, by the computer, at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features;
combining, by the computer, at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features; and,
training, by the computer, at least one classifier using at least some of the first set of combined features, thereby generating the model for prognosing the CV outcome for the monitored subject infected with the SARS-CoV-2.

5. The method of claim 4, wherein the plurality of dynamic and static clinical parameters differs between at two of the reference subjects.

6. The method of claim 4, wherein one or more of the data values in the first set of data values is absent for one or more of the plurality of reference subjects.

7. The method of claim 4, comprising adding one or more additional values to the first set of data values and/or one or more additional dynamic and static clinical parameters to the training database and updating the model for prognosing the CV outcome.

8. The method of claim 4, comprising adding a second set of data values of a second plurality of dynamic and static clinical parameters associated with at least a second plurality of reference subjects infected with the SARS-CoV-2 to the training database and updating the model for prognosing the CV outcome.

9. The method of claim 4, comprising updating the model for prognosing the CV outcome in substantially real-time.

10. (canceled)

11. The method of claim 4, wherein the first plurality of dynamic and static clinical parameters comprises one or more time-series variables.

12.-14. (canceled)

15. The method of claim 4, wherein the dynamic clinical parameters comprise one or more time series features selected from the group consisting of: a short feature, a long feature, and an exponentially weighted decaying feature.

16. The method of claim 15, wherein the short feature comprises a selected period of time prior to a given time point.

17. The method of claim 15, wherein the long feature comprises an entire period to time during which a given reference subject is monitored, wherein corresponding data values are un-weighted.

18. The method of claim 15, wherein the exponentially weighted decaying feature comprises an entire period to time during which a given reference subject is monitored, wherein corresponding data values are weighted.

19.-22. (canceled)

23. The method of claim 4, comprising using the model for prognosing the CV outcome to prognose at least one CV outcome of a monitored test subject infected with the SARS-CoV-2 at one or more time points to produce at least one prognosed test subject CV outcome.

24. The method of claim 23, comprising determining at least one test risk score for the test subject at the one or more time points, wherein a given test risk score that exceeds a predetermined threshold risk score indicates a probability of the test subject experiencing the CV outcome in a given time window beyond the one or more time points.

25. The method of claim 24, comprising determining the test risk score for the test subject in substantially real time.

26. The method of claim 24, comprising repeatedly updating the test risk score for the test subject during at least one selected period of time.

27. The method of claim 24, comprising integrating the test risk score into an electronic health record (EHR) for the test subject.

28. The method of claim 23, comprising administering one or more therapies to the monitored test subject in view of the prognosed test subject CV outcome.

29. (canceled)

30. The method of claim 4, wherein the variable selection algorithm is selected from the group consisting of: a supervised machine learning algorithm, an unsupervised machine learning algorithm, Incremental Association Markov Blanket algorithm, a Grow-Shrink algorithm, and a Semi-Interleaved Hiton-PC algorithm.

31.-36. (canceled)

37. A system, comprising at least one controller that comprises, or is capable of accessing, computer readable media comprising non-transitory computer executable instructions which, when executed by at least one electronic processor, perform at least:
generating a first set of data values of a first plurality of dynamic clinical parameters associated with at least a first plurality of monitored reference subjects infected with severe acute respiratory syndrome coronavirus-2 (SARS-CoV-2), wherein at least a subset of the first set of data values comprises one or more time-series data values;
processing at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features;
combining at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features; and,
training, by the computer, at least one classifier using at least some of the first set of combined features, thereby generating the model for prognosing a cardiovascular (CV) outcome for the monitored subject infected with SARS-CoV-2.

38.-41. (canceled)

42. A computer readable media comprising non-transitory computer executable instruction which, when executed by at least one electronic processor perform at least:
generating a first set of data values of a first plurality of dynamic clinical parameters associated with at least a first plurality of monitored reference subjects infected with severe acute respiratory syndrome coronavirus-2 (SARS-CoV-2), wherein at least a subset of the first set of data values comprises one or more time-series data values;
processing at least some of the first set of data values for at least some of the first plurality of monitored reference subjects infected with the SARS-CoV-2 using one or more sliding time windows that comprise one or more feature time windows associated with one or more outcome time windows, wherein the feature time windows comprise one or more time series features selected from the group consisting of: a short feature, a long feature, and an exponentially weighted decaying feature to produce at least a first set of processed dynamic features;
combining at least some of the first set of processed dynamic features with a second set of data values of a first plurality of static clinical parameters associated with at least some of the first plurality of monitored reference subjects infected with the SARS-CoV-2 for one or more of the time windows to produce at least a first set of combined features; and,
training, by the computer, at least one classifier using at least some of the first set of combined features, thereby generating the model for prognosing a cardiovascular (CV) outcome for the monitored subject infected with SARS-CoV-2.

43. (canceled)
